(19) United States
(12) Patent Application Publication
(10) Pub. No.: US 2023/0357312 A1
(43) Pub. Date: Nov. 9, 2023

(54) Borylated Di-Peptide Amino Acid compositions for use in Boron Neutron Capture Therapy and methods thereof

(71) Applicant: TAE Life Sciences, Foot Hill Ranch, CA (US)

(72) Inventors: Michael Y. Torgov, Redondo Beach, CA (US); Tioga J. Martin, Los Angeles, CA (US); Arthur B. Raitano, Los Angeles, CA (US); Jason C. Quintana, Santa Monica, CA (US); Kendall Morrison, Santa Monica, CA (US)

(21) Appl. No.: 17/803,836

(22) Filed: Dec. 16, 2022

Related U.S. Application Data
(60) Provisional application No. 63/361,403, filed on Dec. 16, 2021.

(51) Int. Cl. C07K 1/107; A61N 5/10

(52) U.S. Cl. CPC C07K 1/1077; A61N 5/10; A61N 2005/1087

(57) ABSTRACT

Borylated Di-Peptide Amino Acid (“Bdi-AA”) compositions and Di-Peptide Amino Acid (di-AA) compositions and methods of making Bdi-AAs and di-AAs are disclosed herein. Consequently, the Bdi-AAs and di-AAs can be administered to patients as a Neutron Capture Agent and provide a method of treating cancer, immunological disorders, and other disease by utilizing a Neutron Capture Therapy modality.

### CROSS-REFERENCE TO RELATED APPLICATIONS

This application claims priority to U.S. Provisional Pat. Application No. 63/361,403 filed 16 Dec. 2021, the contents of which are fully incorporated by reference herein.

### STATEMENT OF RIGHTS TO INVENTIONS MADE UNDER FEDERALLY SPONSORED RESEARCH

Not applicable.

### FIELD OF THE INVENTION

The invention described herein relates to the field of boron neutron capture therapy (BNCT) and related therapies. Specifically, the invention relates to borylated di-peptide amino acid compositions which can be used as a vehicle for neutron capture therapy in humans. The invention further relates to the treatment of cancers and other immunological disorders and diseases.

### BACKGROUND OF THE INVENTION

Cancer is the second leading cause of death next to coronary disease worldwide. Millions of people die from cancer every year and in the United States alone cancer kills well over a half-million people annually, with 1.8 M new cancer cases diagnosed in 2020 (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. In particular, carcinomas of the lung (18.4% of all cancer deaths), breast (6.6% of all cancer deaths), colorectal (9.2% of all cancer deaths), liver (8.2% of all cancer deaths), and stomach (8.2% of all cancer deaths) represent major causes of cancer death for both sexes in all ages worldwide (GLOBOCAN 2018). These and virtually all other carcinomas share a common lethal feature in that they metastasis to sites distant from the primary tumor and with very few exceptions, metastatic disease fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Furthermore, many cancer patients experience a recurrence of their disease.

Although cancer therapy has improved over the past decades and survival rates have increased, the heterogeneity of cancer still demands new therapeutic strategies utilizing a plurality of treatment modalities. This is especially true in treating solid tumors at anatomical crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients’ quality of life.

Neutron Capture Therapy (NCT) is a promising form of radiation therapy. It is a technique that selectively kills tumor cells using boron compound while sparing the normal cells. BNCT relies on the propensity of non-radioactive ¹⁰B isotope to absorb epithermal neutrons that fall into the low energy range of 0.5 keV < Eₙ < 30 keV. Following neutron capture, the boron atom undergoes a nuclear fission reaction giving rise to an alpha-particle and a recoiled lithium nucleus (⁷Li) as follows:

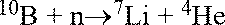

The alpha particle deposits high energy i.e., 150 keV/µm along their short path essentially restricted to a single cell diameter that results in a double strand DNA break followed by cancer cell death by apoptosis. Thus, BNCT integrates a concept of both chemotherapy, targeted therapy, and the gross anatomical localization of traditional radiotherapy.

Even though the conceptual techniques of NCT and specifically Boron Neutron Capture Therapy (BNCT) are well known, the technological limitations associated with this type of treatment have slowed progress. During the early investigations using the research reactors of MIT in 1960′s, several dozens of patients were treated using disodium decahydrodecaborate, which was considered less toxic than simple boron compounds used previously yet capable of delivering more boron to the cell. Unfortunately, BNCT studies were halted in the USA due to the severe brain necrosis in the patients undergoing BNCT and the potential harm of using nuclear reactors.

Hiroshi Hatanaka in 1968 re-investigated clinical application of BNCT in Japan using sodium borocaptate (BSH) by directing the beam to surgically exposed intracranial tumor and reported of achieving 58% of 5-year survival rate. In 1987 clinicians in Japan applied BNCT for the treatment of malignant melanoma using boronophenylalanine (BPA) as boron compound. Thus, slow resurgence of BNCT took place albeit limited to the countries with an access to research reactor facilities capable of delivering epithermal neutron beam. Currently, given the technological improvements in both (i) the infusion and delivery of a capture compound, which preferably concentrates in the tumor, and (ii) more abundant and easier access to neutron beam using cyclotrons, there has been a resurgence in NCT treatment methods.

The proton boron fusion reaction relies on the naturally abundant ¹¹B isotope rather than ¹⁰B required for BNCT. Unlike BNCT, three alpha particles are emitted after the fusion reaction between a proton (¹H) and a boron (¹¹B) nucleus: p+¹¹B -> 3α. The proton beam has the advantage of a Bragg-peak characteristic reducing the normal tissue damage and when combined with proton capture, may improve the efficacy of the proton therapy alone.

Carriers of boron have evolved since 1950s and are reviewed in NEDUNCHEZHIAN, et. al., J. Clin. & Diag. Res., vol. 10(12) pp. ZE01-ZE04 (December 2016). Briefly, the 1^{st} generation of boron compounds represented by boric acid and its derivatives were either toxic or suffered from low tumor accumulation/retention. BPA and BSH are both considered the 2^{nd} generation compounds that emerged in 1960s. These had significantly lower toxicity and better PK and biodistribution. BPA-fructose complex is considered the 3^{rd} generation compound that is used to treat patients with H&N, glioblastoma and melanoma using BNCT since 1994. BPA-fructose and BSH are the only compounds that are being used in clinic as boron carriers to date although both low and high molecular weight biomolecules such as nucleosides, porphyrins, liposomes, nanoparticles and mAbs have been evaluated for the tumor targeting in preclinical models. The main deficiency of BPA-fructose is relatively low solubility combined with its rapid clearance that prevents achieving high Cₘₐₓ in blood, one of the drivers influencing the tumor uptake.

From the aforementioned, it will be readily apparent to those skilled in the art that a new treatment paradigm is needed in the treatment of cancers and immunological diseases. By using modern chemical synthesis and modifying natural amino acids with boron, a new disease treatment can be achieved with the overall goal of more effective treatment, reduced side effects, and lower production costs.

Given the current deficiencies associated with NCT, it is an object of the present invention to provide new and improved methods of treating cancer(s), immunological disorders, and other diseases utilizing borylated amino acids as a capture agent in NCT and BNCT treatments.

### SUMMARY OF THE INVENTION

The invention provides for compositions comprising natural di-peptide amino acids which have been borylated via chemical synthesis for use as a delivery modality to treat human diseases such as cancer, immunological disorders, including but not limited to rheumatoid arthritis, ankylosing spondylitis, and other cellular diseases, including but not limited to Alzheimer’s disease. In certain embodiments, the borylated amino acids are comprised of naturally occurring amino acids such as phenylalanine, tryptophan, tyrosine, histidine, and any other naturally occurring amino acid set forth in Table 1.

In a further embodiment, the invention comprises methods of concentrating Boron in a cell comprising (i) synthesizing a borylated di-peptide amino acid (“Bdi-AA”); (ii) administering the Bdi-AA to a patient, and (iii) irradiating the cell with neutrons.

In another embodiment, the present disclosure teaches methods of synthesizing Bdi-AA’s.

In another embodiment, the present disclosure teaches methods of synthesizing BPA-Ala.

In another embodiment, the present disclosure teaches methods of synthesizing His-BPA.

In another embodiment, the present disclosure teaches methods of synthesizing Leu-BPA.

In another embodiment, the present disclosure teaches methods of synthesizing Ala-BPA.

In another embodiment, the present disclosure teaches methods of synthesizing BPA-BPA.

In another embodiment, the present disclosure teaches methods of synthesizing 3-boronobenzyl-BPA.

In another embodiment, the present disclosure teaches methods of synthesizing 3-BPA-boronopyridine.

In another embodiment, the present disclosure teaches methods of synthesizing reduced BPA-BPA (denoted “(Red)-BPA-BPA″).

In another embodiment, the present disclosure teaches methods of synthesizing BPA-Tyr.

In another embodiment, the present disclosure teaches methods of synthesizing Tyr-BPA.

In another embodiment, the present disclosure teaches methods of synthesizing BPA-Leu.

In another embodiment, the present disclosure teaches methods of synthesizing borylated dipeptides having a chemical structure 27 set forth in FIG. 11.

In another embodiment, the present disclosure teaches methods of synthesizing borylated dipeptides having a chemical structure 30 set forth in FIG. 12.

In another embodiment, the present disclosure teaches methods of synthesizing borylated dipeptides having a chemical structure 33 set forth in FIG. 13.

In another embodiment, the present disclosure teaches methods of synthesizing borylated dipeptides having a chemical structure 6, 11, 17, 21, 24, 35, 36, 37, and 38 set forth in FIG. 14.

In another embodiment, the present disclosure teaches methods of synthesizing borylated dipeptides having a chemical structure 36 set forth in FIG. 15.

In another embodiment, the present disclosure teaches methods of synthesizing borylated dipeptides having a chemical structure 37 set forth in FIG. 17.

In another embodiment, the present disclosure teaches methods of synthesizing borylated dipeptides having a chemical structure 38 set forth in FIG. 19.

In another embodiment, the present disclosure teaches methods of treating cancer(s), immunological disorders, and other diseases in humans.

### BRIEF DESCRIPTION OF THE FIGURES

FIG. 1. Chemical Synthesis for BPA-Ala.

FIG. 2. Purity Analysis of BPA-Ala.

FIG. 3. Chemical Synthesis for His-BPA.

FIG. 4. Purity Analysis of His-BPA.

FIG. 5. Chemical Synthesis for Leu-BPA.

FIG. 6. Purity Analysis of Leu-BPA.

FIG. 7. Chemical Synthesis for Ala-BPA.

FIG. 8. Purity Analysis of Ala-BPA.

FIG. 9. Chemical Synthesis for BPA-BPA.

FIG. 10. Purity Analysis of BPA-BPA.

FIG. 11. Chemical Synthesis for 3-boronobenzyl-BPA.

FIG. 12. Chemical Synthesis for BPA-3-boronopyridine.

FIG. 13. Chemical Synthesis for (Red)-BPA-BPA.

FIG. 14. Chemical Structures of Exemplary Dipeptide(s).

FIG. 15. Chemical Synthesis for BPA-Tyr.

FIG. 16. Purity Analysis of BPA-Tyr.

FIG. 17. Chemical Synthesis for Tyr-BPA.

FIG. 18. Purity Analysis of Tyr-BPA.

FIG. 19. Chemical Synthesis for BPA-Leu.

FIG. 20. Purity Analysis of BPA-Leu.

FIG. 21. Uptake of Borylated Dipeptides in Multiple Cancer Cell Lines.

FIG. 22. Correlation of Uptake of BPA and His-BPA and Relative Expression of LAT1 and PEPT1 in Cell Lines.

FIG. 23. Correlation of Uptake of BPA and His-BPA and Relative Expression of LAT1 and PEPT1 in Cell Lines, continued.

FIG. 24. His-BPA Uptake Inhibition by Gly-SAR in PEPT1 Cells.

FIG. 25. Relative Uptake of Dipeptides in PEPT1 Cells.

FIG. 26. Evaluation of Dipeptide Degradation to BPA by HPLC. FIG. 26(A) shows BPA-BPA incubation with FaDu cell; 225.0 nm. FIG. 26(B) shows BPA-BPA control which was incubated with media and no cells; 225.0 nm. FIG. 26(C) shows two (2) QDa positive scans (Peak 1 = 0.513) and (Peak 2 = 2.105).

FIG. 27. Evaluation of Dipeptide Degradation to BPA by HPLC, continued.

FIG. 28. Pharmacokinetics of BPA-BPA.

FIG. 29. Biodistribution of Dipeptides Using FaDu Cells In Vivo.

FIG. 30. Biodistribution of His-BPA Using FaDu Cells In Vivo.

FIG. 31. Dose Escalation of Boron Delivery using Borylated dipeptides in Multiple Xenograft Models In Vivo.

FIG. 32. Measuring Tumor Boron Content of Multiple Dipeptides Using CT26 Syngeneic Colon Cancer Model.

FIG. 33. Improved BNCT Efficiency Using Borylated Dipeptides Compared to BPA. FIG. 33(A) shows 12-minute standard irradiation at 5 MW. FIG. 33(B) shows 6-minute standard irradiation at 5 MW.

FIG. 34. BNCT Tumor Regression Using Borylated Dipeptides. FIG. 34(A) shows multiple borylated di-peptides with control groups. FIG. 34(B) shows only B¹⁰-BPA-BPA and B¹⁰-HIS-BPA at 900 mg/kg.

### DETAILED DESCRIPTION OF THE INVENTION

### Outline of Sections

I.) Definitions
II.) BPA
III.) BSH
IV.) Boron
   a. Boron Generally
V.) Naturally Occurring Amino Acids
VI.) Borylated Di-Peptide Amino Acids (Bdi-AAs)
   a. BPA-Ala
   b. His-BPA
   c. Leu-BPA
   d. Ala-BPA
   e. BPA-BPA
   f. Tyr-BPA
   g. BPA-Tyr
   h. BPA-Leu
   i. Di-Peptide Amino Acid Composition(s)
      i. 3-boronobenzyl-BPA
      ii. BPA-3-boronopyridine
      iii. Reduced (RED)-BPA-BPA
VII.) Boron Neutron Capture Therapy Using Bdi-AAs
VIII.) Proton Boron Fusion Therapy Using Bdi-AAs
IX.) Methods of Delivering Bdi-AAs to a Cell
X.) KITS/Articles of Manufacture

### I.) Definitions

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

The terms “advanced cancer”, “locally advanced cancer”, “advanced disease” and “locally advanced disease” mean cancers that have extended through the relevant tissue capsule and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease and these patients have substantially less favorable outcomes compared to patients having clinically localized (organ-confined) cancer.

“Amino Acid” means a simple organic compound containing both a carboxyl (—COOH) and an amino (—NH₂) group.

“Borylation” means reactions that produce an organoboron compound through functionalization of aliphatic and aromatic C—H bonds.

“Borylated Amino Acid” (BAA) means a compound comprising a naturally occurring amino acid, such as those set forth in Table I, which has undergone a borylation reaction. BAAs can be synthesized in multiple formats depending on the underlying amino acid that is being used.

“Borylated Di-Peptide Amino Acid” (Bdi-AA) means a borylation of an amino acid that is paired with either (i) a non-borylated amino acid, or (ii) a second borylated amino acid. For the purposes of this definition, an amino acid relating to this definition can be from any amino acid set forth in Table I so as to include doubling of amino acids and unique pairs.

The term “compound” refers to and encompasses the chemical compound (e.g. a Bdi-AA) itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above

The terms “inhibit” or “inhibition of” as used herein means to reduce by a measurable amount, or to prevent entirely.

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “metastatic cancer” and “metastatic disease” mean cancers that have spread to regional lymph nodes or to distant sites and are meant to include stage D disease under the AUA system and stage T×N×M+ under the TNM system.

“Molecular recognition” means a chemical event in which a host molecule is able to form a complex with a second molecule (i.e., the guest). This process occurs through non-covalent chemical bonds, including but not limited to, hydrogen bonding, hydrophobic interactions, ionic interaction.

“Pharmaceutically acceptable” refers to a non-toxic, inert, and/or composition that is physiologically compatible with humans or other mammals.

The term “neutron capture agent” means a stable non-reactive chemical isotope which, when activated by neutrons produces alpha particles.

The term “neutron capture therapy” means a noninvasive therapeutic modality for treating locally invasive malignant tumors such as primary brain tumors and recurrent head and neck cancer and other immunological disorders and disease by irradiating a neutron capture agent with neutrons.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

### II.) BPA

By way of reference, (¹⁰B)-BPA, L-BPA, or 4-Borono-L-phenylalanine (Sigma Aldrich, St. Louis, MO) is a synthetic compound with the chemical formula:

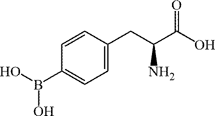 [SMILES: N[C@@H](Cc1ccc(B(O)O)cc1)C(=O)O]

. and is an important boronated compound useful in the treatment of cancer though BNCT. It is a widely known compound which many syntheses have been developed (See, US 8,765,997, Taiwan Biotech Co, Ltd., Taoyuan Hsein, Taiwan, and US2017/0015684, Stella Pharma Corp., Osaka Prefecture Univ., Osaka, Japan)

### III.) BSH

In addition, BSH, or sodium borocaptate, or BSH sodium borocaptate, or Borocaptate sodium B10, or un-decahydrododecaborane thiol is a synthetic chemical compound with the chemical formula:

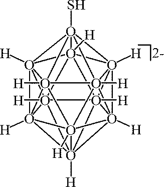

where boron atoms are represented by dots in the vertices for the ecosahedron. BSH is used . as a capture agent in BNCT. Generally speaking, BSH is injected into a vein and becomes concentrated in tumor cells. The patient then receives radiation treatment with atomic particles called neutrons. The neutrons fuse with the boron nuclei in BSH and to produce high energy alpha particles that kill the tumor cells.

### IV. Boron

### (a.) Boron Generally

Generally speaking, and for purposes of this disclosure, Boron is a chemical element with symbol B and atomic number five (5). Primarily used in chemical compounds, natural boron is composed of two stable isotopes, once of which is Boron-10 and the other is Boron-11. Boron-10 isotope is useful for capturing epithermal neutrons, which makes it a promising tool in a therapeutic context using Boron Neutron Capture Therapy. Biologically, the borylated compounds disclosed herein are nontoxic to humans and animals. Based on the foregoing, it will be readily apparent to one of skill in the art that improved modalities for providing high concentrations of boron into a cancer cell are advantageous. It is an object of the present disclosure to provide that advantage.

### V.) Naturally Occurring Amino Acids

Generally speaking, and for the purposes of this disclosure, naturally occurring amino acids are organic compounds containing amine (—NH₂) and carboxyl (—COOH) functional groups, along with a side chain (R group) specific to each amino acid. The key elements of an amino acid are carbon (C), hydrogen (H), oxygen (0), and nitrogen (N), although other elements are found in the side chains of certain amino acids. About 500 naturally occurring amino acids are known (though only 20 appear in the genetic code (Table I)) and can be classified in many ways. They can be classified according to the core structural functional groups’ locations as alpha- (a-), beta- (β-), gamma- (y-) or delta- (δ-) amino acids; other categories relate to polarity, pH level, and side chain group type (aliphatic, acyclic, aromatic, containing hydroxyl or sulfur, etc.). In the form of proteins, amino acid residues form the second-largest component (water is the largest) of human muscles and other tissues. Beyond their role as residues in proteins, amino acids participate in a number of processes such as neurotransmitter transport and biosynthesis.

The twenty (20) amino acids encoded directly by the genetic code (See, Table I) can be divided into several groups based on their properties. Principal factors are charge, hydrophilicity or hydrophobicity, size, and functional groups. These properties are important for protein structure and protein-protein interactions. The water-soluble proteins tend to have their hydrophobic residues (Leu, lle, Val, Phe, and Trp) buried in the middle of the protein, whereas hydrophilic side chains are exposed to the aqueous solvent.

The integral membrane proteins tend to have outer rings of exposed hydrophobic amino acids that anchor them into the lipid bilayer. In the case part-way between these two extremes, some peripheral membrane proteins have a patch of hydrophobic amino acids on their surface that locks onto the membrane. In similar fashion, proteins that have to bind to positively charged molecules have surfaces rich with negatively charged amino acids like glutamate and aspartate, while proteins binding to negatively charged molecules have surfaces rich with positively charged chains like lysine and arginine. There are different hydrophobicity scales of amino acid residues.

Some amino acids have special properties such as cysteine, which can form covalent disulfide bonds to other cysteine residues, proline that forms a cycle to the polypeptide backbone, and glycine that is more flexible than other amino acids.

### VI.) Borylated Di-Peptide Amino Acids (Bdi-AAs)

Based on the foregoing, those of ordinary skill in the art have shown that essential amino acid transporter proteins such as LAT1 are responsible for the uptake of certain naturally occurring amino acids. See, INDIVERI, et. al., Frontiers in Chem., Vol. 6, Art. 243 (June 2018). Notably, the large neutral amino acid transporter 1 (LAT-1, SLC7a5) is a sodium- and pH-independent transporter, which supplies essential amino acids (e.g., leucine, phenylalanine) to cells. The actual functional transporter is a heterodimeric disulfide-linked complex composed of the multi-transmembrane subunit SLC7a5 and single transmembrane subunit SLC3a2 (CD98). LAT-1 is the main transporter to channel essential amino acids across such compartments as placenta or blood brain barrier. LAT1 also transports the thyroid hormones T3 and T4, the dopamine precursor L-DOPA, as well as amino acid-related exogenous compounds, such as the drugs melphalan and gabapentin. See, FRIESEMA, et. al., Endocrinology, 142(10): pp. 4339-4348 (October 2001) and UCHINO, et. al., Mol. Pharmacol, 61:729-737 (2002).

Moreover, its expression is highly upregulated in several types of human cancer that are characterized by an intense demand for amino acids for metabolism and growth. See, SINGH, et. al., Int. J. Mol. Sci., 19, 1278 (24-April-2018). Furthermore, it has been reported that the nature of the amino acid side chain influences selectivity of LAT1 for various amino acids, with the following order in terms of increasing rate of transport: Phe > Trp > Leu > lle > Met > His > Tyr > Val. See, KANAI, et. al., J. Bio. Chem., Vol. 273, No. 37, pp. 23629-23632 (Sept. 11, 1998). However, the influence of a boron addition modification to amino acids is not believed to have been previously taught.

It should be noted that 4-Borono-L-phenylalanine (L-BPA), has been used in numerous clinical studies using BNCT and is approved for use in Japan for the treatment of squamous head and neck carcinomas, in conjunction with a Sumitomo accelelrator as the neutron source. L-BPA is shown to be safe, stable, and can be readily manufactured. However, despite these successes, it has several significant drawbacks. These drawbacks include its poor solubility (1.7 mg/mL) and reliance on fructose formulation in order to achieve a yield of 30 mg/mL. See, Watanabe et al., BMC Cancer 16 :859 (2016). It is known that such a concentrated solution is quasi-stable and must be ready for immediate use due to a high risk of precipitation.

In addition, commercially available di-peptide compositions are available for use in the manufacturing of cell cultures (GlutaMAX, Thermo-Fisher, Waltham, MA). With this principle in mind, the present disclosure contemplates the synthesis of naturally occurring di-peptide amino acids through borylation reactions to create Borylated Di-Peptide Amino Acids (“Bdi-AAs”) with tumor seeking and tumor localizing properties for use as neutron capture agent in Boron Neutron Capture Therapy (“BNCT”) and/or Boron Proton Capture Therapy. See, for example, ROBERTS, et. al., Tetrahedron Letters, vol. 21, Issue 36, pp. 3435-3438 (1980).

Borylated dipeptides are considerably more soluble than BPA and are taken up by cancer cells both in vitro and in vivo. The mechanism of the intracellular uptake of the borylated dipeptides is not understood but it likely involves, at least in part, a proteolytic cleavage by a surface peptidase to release L-BPA which is then transported through LAT-1.

In the alternative, an oligopeptide transporter e.g., PEPT-1 (SLC15) or PEPT-2 may be involved, as is seen in the gastrointestinal tract. See GONG, et. al., Oncotarget, Vol. 8, (No. 25), pp: 40454-40468 (2017) and MIYABE, et. al., J Pharmacol. Sci., 139(3):215-222 (2019).

Based on the aforementioned rationale, in this disclosure specifically designed borylated oligopeptides were evaluated for in vitro uptake using various cancer cell lines. Additionally, it is taught in this disclosure that established sub-cutaneous xenografts models in mice were used to determine pharmacokinetics and biodistribution of boron in a tumor, blood, and other organs.

The therapeutic potential of BNCT rests in the selective accumulation of a sufficient amount of ¹⁰B within cancer cells. To investigate the ability of borylated dipeptides to deliver ¹⁰B boron to cancer cells, the disclosure teaches a synthesized panel of dipeptides and interrogated a range of concentrations illustrating what is believed to be the physiologically relevant amounts for boronophenylalanine (BPA), currently the most widely studied boron drug in BNCT clinical practice.

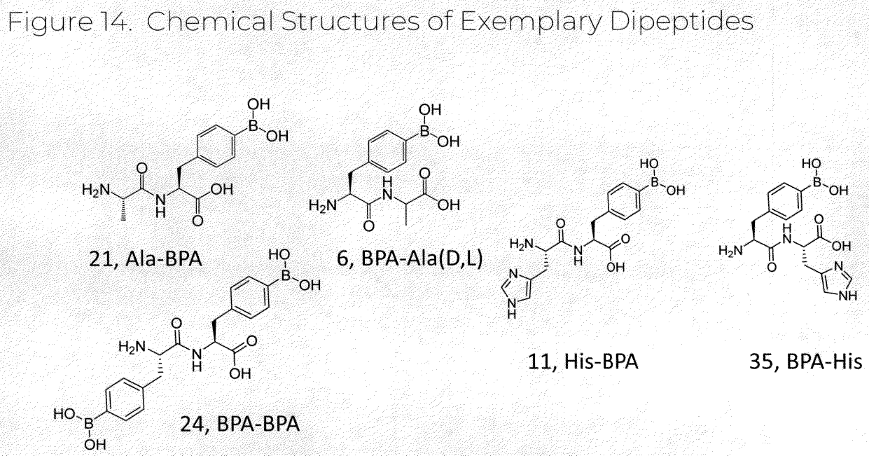

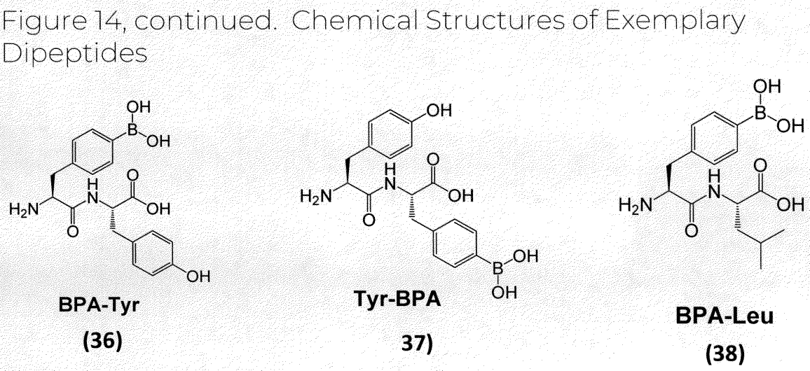

Based on the above-referenced background and therapeutic rationale, the following Bdi-AAs are disclosed herein and more fully referenced in FIG. 14. Specific characterization of the chemical structures are more fully set forth below:

### (a) BPA-Alanine (i.e., BPA-Ala)

In one embodiment, a composition with the following formula is within the scope of the of the present disclosure:

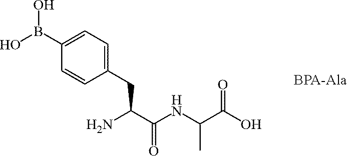 [SMILES: CC(NC(=O)[C@@H](N)Cc1ccc(B(O)O)cc1)C(=O)O]

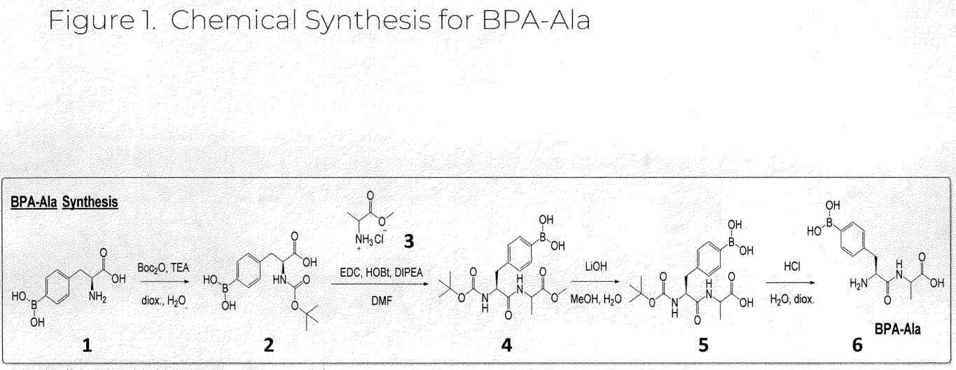

It will be appreciated by one of ordinary skill in the art that the above composition is synthesized as shown in FIG. 1.

### (b) Histidine-BPA (i.e., His-BPA)

In one embodiment, a composition with the following formula is within the scope of the of the present disclosure:

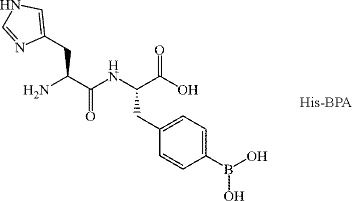 [SMILES: N[C@@H](Cc1c[nH]cn1)C(=O)N[C@@H](Cc1ccc(B(O)O)cc1)C(=O)O]

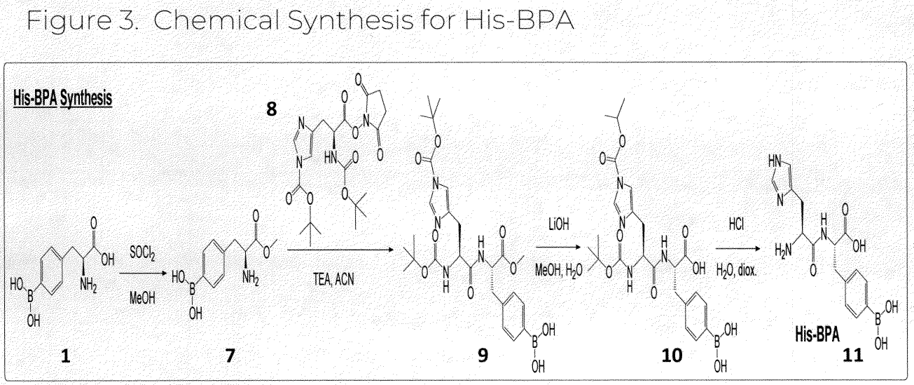

It will be appreciated by one of ordinary skill in the art that the above composition is synthesized as shown in FIG. 3.

### (c) Leucine-BPA (i.e., Leu-BPA)

In one embodiment, a composition with the following formula is within the scope of the of the present disclosure:

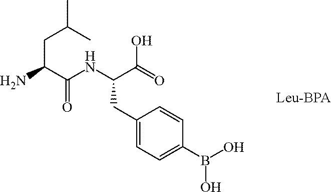 [SMILES: CC(C)C[C@H](N)C(=O)N[C@@H](Cc1ccc(B(O)O)cc1)C(=O)O]

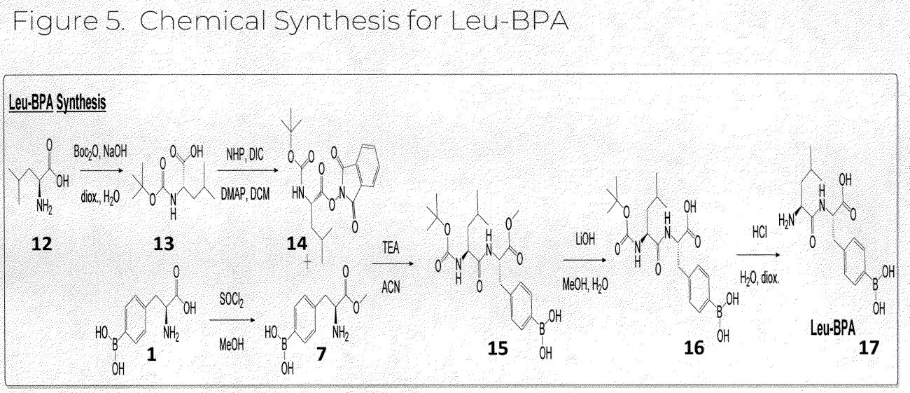

It will be appreciated by one of ordinary skill in the art that the above composition is synthesized as shown in FIG. 5.

### (d) Alanine-BPA (i.e., Ala-BPA)

In one embodiment, a composition with the following formula is within the scope of the of the present disclosure:

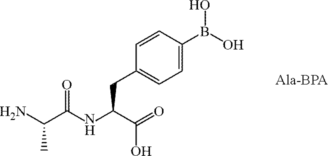 [SMILES: C[C@H](N)C(=O)N[C@@H](Cc1ccc(B(O)O)cc1)C(=O)O]

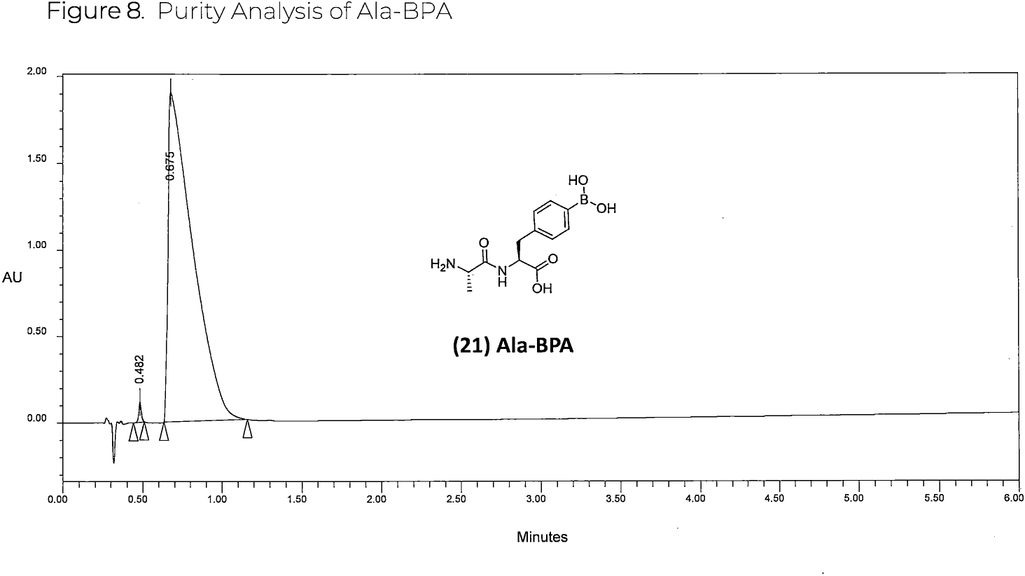

It will be appreciated by one of ordinary skill in the art that the above composition is synthesized as shown in FIG. 7.

### (e) BPA-BPA

In one embodiment, a composition with the following formula is within the scope of the of the present disclosure:

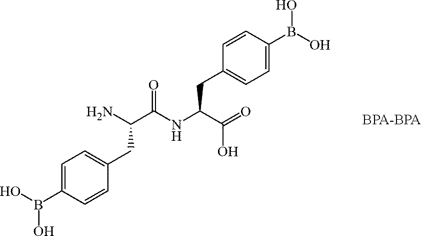 [SMILES: N[C@@H](Cc1ccc(B(O)O)cc1)C(=O)N[C@@H](Cc1ccc(B(O)O)cc1)C(=O)O]

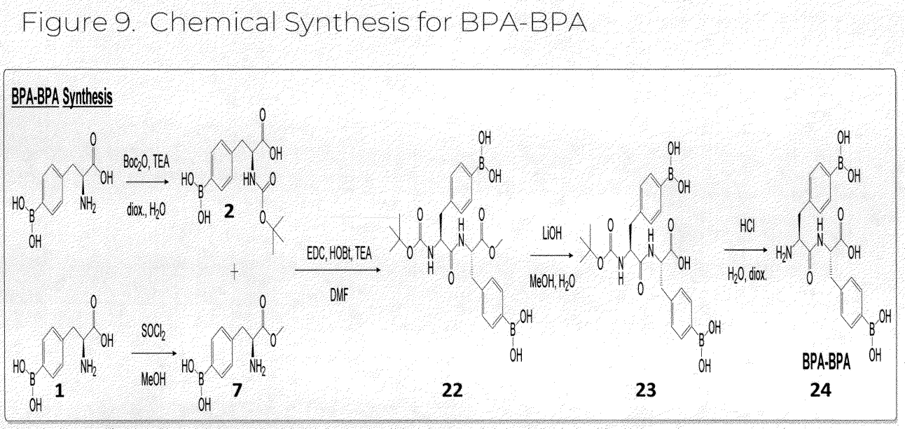

It will be appreciated by one of ordinary skill in the art that the above composition is synthesized as shown in FIG. 9.

### (f) Tyrosine-BPA, I.e., Tyr-BPA

In one embodiment, a composition with the following formula is within the scope of the of the present disclosure:

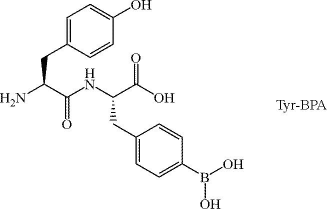 [SMILES: N[C@@H](Cc1ccc(O)cc1)C(=O)N[C@@H](Cc1ccc(B(O)O)cc1)C(=O)O]

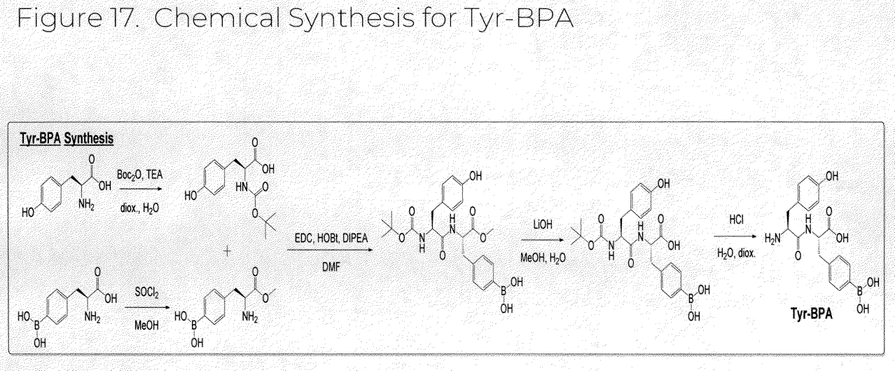

It will be appreciated by one of ordinary skill in the art that the above composition is synthesized as shown in FIG. 17.

In one embodiment, a composition with the following formula is within the scope of the of the present disclosure:

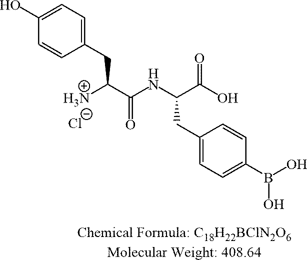

See also, MIYABE, et. al., J. of Pharmacological Sciences 139 (2019) pp. 215-222.

### (g) BPA-Tyrosine, I.e., BPA-Tyr

In one embodiment, a composition with the following formula is within the scope of the of the present disclosure:

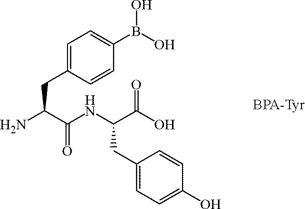 [SMILES: N[C@@H](Cc1ccc(B(O)O)cc1)C(=O)N[C@@H](Cc1ccc(O)cc1)C(=O)O]

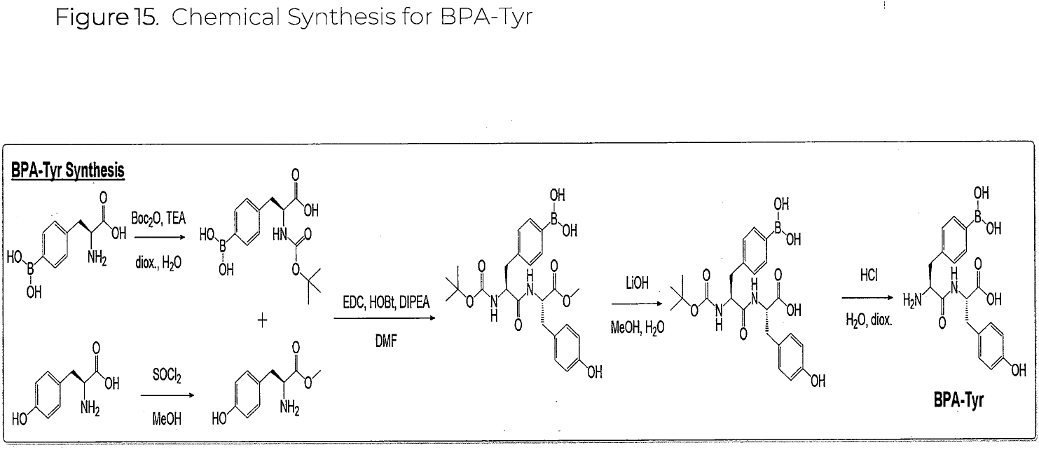

It will be appreciated by one of ordinary skill in the art that the above composition is synthesized as shown in FIG. 15.

### (h) BPA-Leucine, I.e., BPA-Leu

In one embodiment, a composition with the following formula is within the scope of the of the present disclosure:

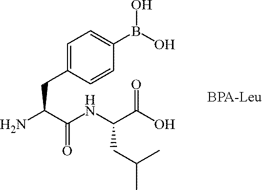 [SMILES: CC(C)C[C@H](NC(=O)[C@@H](N)Cc1ccc(B(O)O)cc1)C(=O)O]

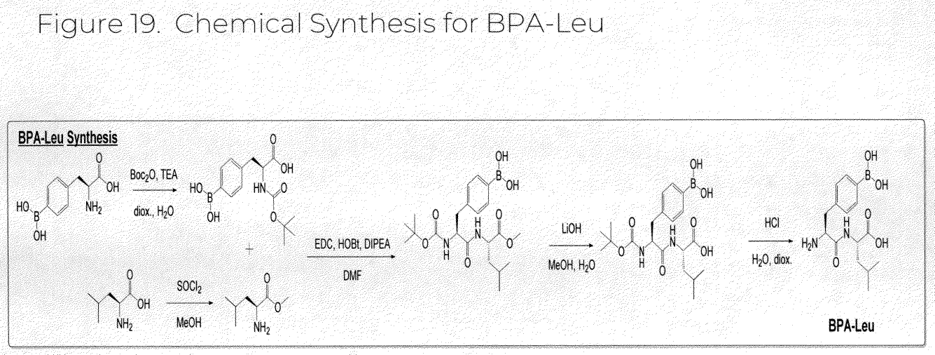

It will be appreciated by one of ordinary skill in the art that the above composition is synthesized as shown in FIG. 19.

It will be appreciated by one of ordinary skill in the art that the synthesis of these compounds can be achieved through the nucleophilic addition of BPA to the Boc protected succinimic ester functionalized BPA, followed by deprotection of the Boc group, peptide coupling between Fmoc-protected BPA and ammonia undecahydrododecaborate followed by deprotection to reveal the target material.

Utilizing the compositions of the present disclosure, Bdi-AA’s which have a functional uptake in certain complexes can be synthesized to deliver concentrated amounts of boron to a cancer or otherwise diseased cell for use in BNCT and/or other cancer treatment modalities. Based on the ability for certain antigen complexes to upregulate specific amino acids, various branched-chain and aromatic amino acids can be explored. For purposes of this disclosure, in one embodiment, the amino acid comprises valine, leucine, isoleucine, histidine, tryptophan, tyrosine, and any amino acid set for in Table I and/or borylated amino acids from the amino acids set forth in Table I.

The principle can be achieved through side chain manipulations, peptide couplings, and decarboxylation-borylation. See, LI, et. al., Science 356, 1045 (2017), and MALAN, et. al., Synlett 1996(02): 167-168; and US2018/0155368 (Neuboron Medtech, Nanjing, China). The wide diversity of useful reactivity that is specific to boronic acids such as cross-coupling, oxidation, amination, and homologation is shown to guide retrosynthetic analysis. Also contemplated in the present disclosure is the manipulation of solubility and lipophilicity through the use of boronic esters in lieu of acid. Subsequent to the modification(s) disclosed herein, additional antigen complexes and transporters may be implicated through selective borylation of their respective molecular substrates.

### (i) Di-Peptide Composition(s)

In one embodiment, a Bdi-AA with the following formula is within the scope of the of the present disclosure:

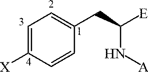

Wherein,
A = H, Amino Acid, or Borolated Amino Acid;
E = CO₂H, B(OH)₂, CO-Amino Acid, or CO-Borolated Amino Acid, CO—NHB₁₂H₁₁; and
X = H, B(OH)₂, or B(OR)₂, but can be at 2, 3, or 4 position.

In a further embodiment, a Bdi-AA with the following formula is within the scope of the of the present disclosure:

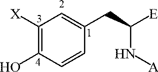

Wherein,
A = H, Amino Acid, or Borylated Amino Acid;
E = CO₂H, B(OH)₂, CO-Amino Acid, or CO-Borylated Amino Acid, CO—NHB₁₂H₁₁; and
X = H, B(OH)₂, or B(OR)₂, but can be at 2, or 3 position.

In a further embodiment, a Bdi-AA with the following formula is within the scope of the of the present disclosure:

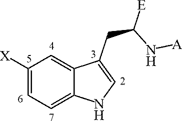

Wherein,
A = H, Amino Acid, or Borylated Amino Acid;
E = CO₂H, B(OH)₂, CO-Amino Acid, or CO-Borylated Amino Acid, CO—NHB₁₂H₁₁; and
X = H, B(OH)₂, or B(OR)₂, but can be at 2, 4, 5, 6, or 7 position.

### (i) 3-boronobenzyl-BPA

In one embodiment, a composition with the following formula is within the scope of the of the present disclosure:

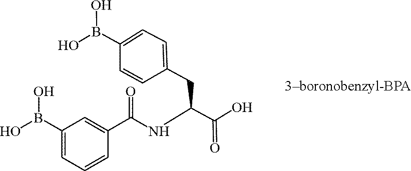 [SMILES: O=C(N[C@@H](Cc1ccc(B(O)O)cc1)C(=O)O)c1cccc(B(O)O)c1]

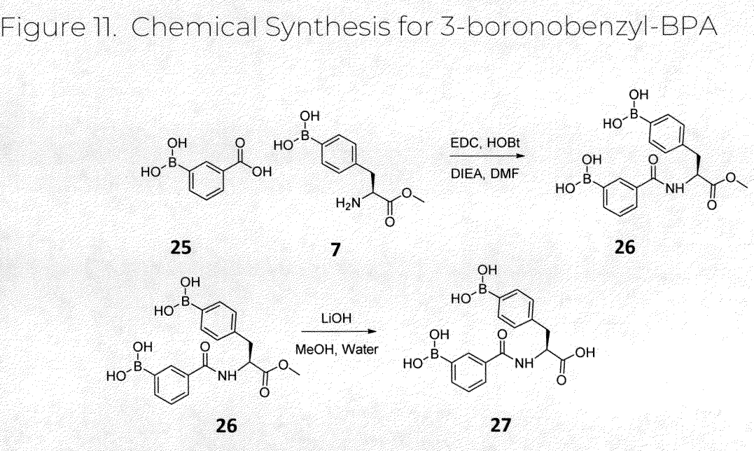

It will be appreciated by one of ordinary skill in the art that the above composition is synthesized as shown in FIG. 11.

### (ii) BPA-3-boropyridine

In one embodiment, a composition with the following formula is within the scope of the of the present disclosure:

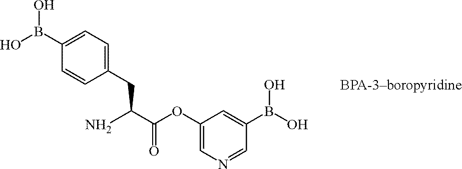 [SMILES: N[C@@H](Cc1ccc(B(O)O)cc1)C(=O)Oc1cncc(B(O)O)c1]

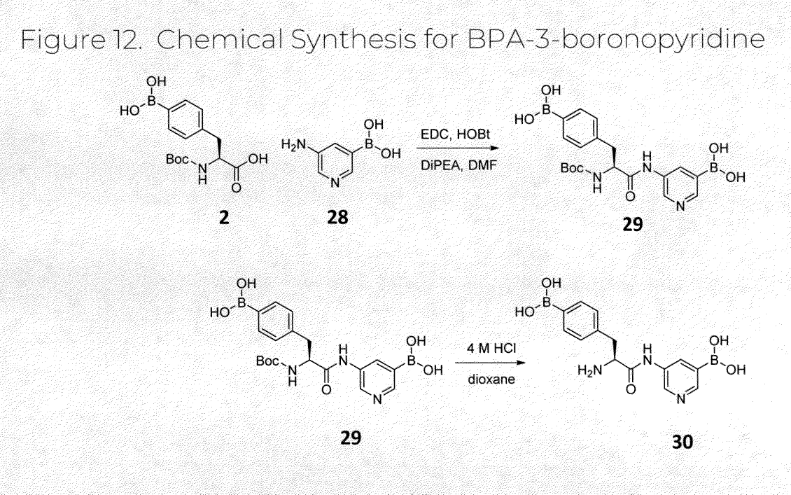

It will be appreciated by one of ordinary skill in the art that the above composition is synthesized as shown in FIG. 12.

### (iii) Reduced (RED)-BPA-BPA

In one embodiment, a composition with the following formula is within the scope of the of the present disclosure:

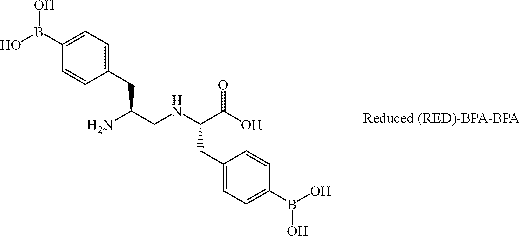 [SMILES: N[C@H](CN[C@@H](Cc1ccc(B(O)O)cc1)C(=O)O)Cc1ccc(B(O)O)cc1]

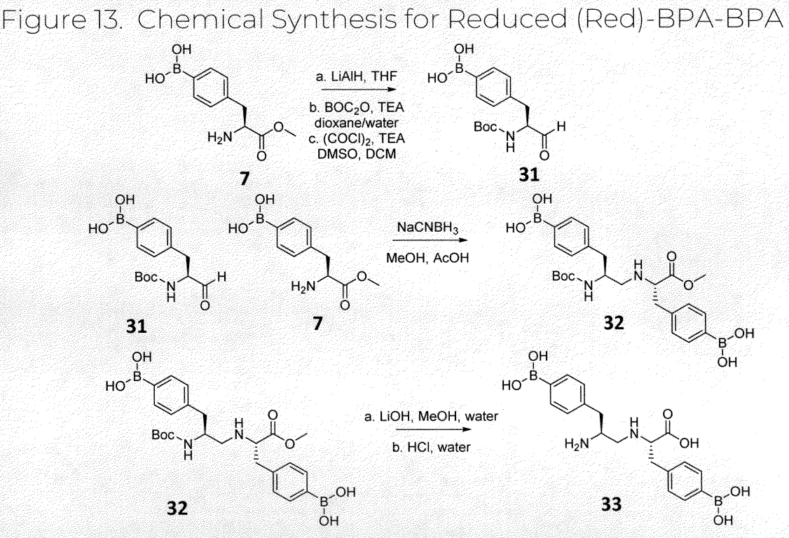

It will be appreciated by one of ordinary skill in the art that the above composition is synthesized as shown in FIG. 13.

It will be appreciated by one of skill in the art that various modifications to the aforementioned structures can be readily made using methods in the art. Such modifications or other embodiments can be practiced without departing from the true scope and spirit of the invention.

### VII.) Boron Neutron Capture Therapy Using Bdi-AAs

One aspect of the present disclosure is the use of Bdi-AAs as a modality for Boron Neutron Capture Therapy (BNCT) and/or Boron Proton Capture Therapy (“BPCT”). Briefly, BNCT is a binary treatment modality in which neither component alone is lethal or toxic to the tumor. The two components comprise (i) the infusion or delivery of a capture compound, which preferentially is concentrated in the tumor, and (ii) the irradiation of the tumor site by neutrons or by protons. In BNCT, given the large cross-section of thermal neutron interactions with ¹⁰B, there is consequently a high probability of a splitting of Boron nucleus into ⁴He²⁺ and ⁷Li⁺. Given that the ionization capability of He²⁺ and Li⁺ is high, and the distances travelled are short, then the cells preferably enriched by Boron are killed and the healthy cells are damaged much less due to the lack of high concentration of boron. Given this, the advantage of BNCT is the destruction of tumor cells without a highly traumatic surgical procedure. However, as will be understood by one of skill in the art, success is predicated high concentration and selective localization of ¹⁰B in tumor cells.

In one embodiment, ¹⁰B is concentrated on a Bdi-AA. The Bdi-AA is then given to a patient and the Bdi-AA is localized into a tumor cell. The Bdi-AA containing ¹⁰B are concentrated into the tumor and the tumor is irradiated using epithermal neutrons. The tumor cells are destroyed.

### VIII.) Proton Boron Fusion Therapy Using Bdi-AAs

Another aspect of the present disclosure is the use of Bdi-AAs as a modality for Proton Boron Fusion Therapy (PBFT). Briefly, the proton boron fusion reaction was introduced in the 1960s. Three alpha particles are emitted after the reaction between a proton (¹H) and a boron particle (¹¹B). These three alpha particles provide the damage to the tumor cell, just as in the case of alpha particles in BNCT. Theoretically, in the case of PBFT, the therapy efficacy per incident particle is three times (3×) greater than that of BNCT. In addition, because the proton beam has the advantage of a Bragg-peak characteristic, normal tissue damage can be reduced. Generally speaking, many studies for tumor treatment using alpha particles have been performed. In order to take advantage of alpha particles for dose delivery, two key points should be considered. First, the boron uptake should be labeled accurately to the target cell. As mentioned previously, alpha particles are generated where the boronated compound is accumulated. If this happens in normal tissue near the tumor region, alpha particles will damage the normal tissue as well as the tumor cell. Second, the number of generated alpha particles is also a significant factor for effective therapy. By using PBFT, a more effective therapy can be realized compared to BNCT or conventional proton therapy alone.

In one embodiment, ¹⁰B and/or ¹¹B is concentrated on a Bdi-AA. The Bdi-AA is then given to a patient and the Bdi-AA is localized into a tumor cell. The Bdi-AA containing ¹⁰B and/or ¹¹B are concentrated into the tumor and the tumor is irradiated using epithermal neutrons. The tumor cells are destroyed.

### IX.) Methods of Delivering Bdi-AAs to a Cell

As will be appreciated by one of ordinary skill in the art, the ability to efficiently deliver high concentrations of Boron to a cell is an advantage of the present invention.

It is shown that the Bdi-AAs of the present disclosure enables a higher amount of boron to be administered to a cell safely in mammals. Briefly, Bdi-AAs of the disclosure are prepared as set forth in the disclosure. The resulting Bdi-AA are taken up by the tumor cell by the upregulated LAT1 transporter protein and/or an upregulated dipeptide transporter protein such as PEPT1.

### X.) Kits/Articles of Manufacture

For use in the laboratory, prognostic, prophylactic, diagnostic and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise a Bdi-AA or several Bdi-AAs of the disclosure. Kits can comprise a container comprising a drug unit. The kit can include all or part of the Bdi-AAs and/or diagnostic assays for detecting cancer and/or other immunological disorders.

The kit of the invention will typically comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic or laboratory application, and can also indicate directions for either in vivo or in vitro use, such as those described herein. Directions and or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as a cancer or other immunological disorder.

The terms “kit” and “article of manufacture” can be used as synonyms.

In another embodiment of the invention, an article(s) of manufacture containing compositions, such as Bdi-AAs of the disclosure. The article of manufacture typically comprises at least one container and at least one label. Suitable containers include, for example, bottles, vials, syringes, and test tubes. The containers can be formed from a variety of materials such as glass, metal, or plastic. The container can hold one or several Bdi-AAs and/or one or more therapeutics doses of Bdi-AAs.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be a Bdi-AA of the present disclosure.

The article of manufacture can further comprise a second container comprising a pharmaceutically acceptable buffer, such as phosphate-buffered saline, Ringer’s solution and/or dextrose solution. It can further include other materials desirable from a commercial and user standpoint, including other buffers, diluents, filters, stirrers, needles, syringes, and/or package inserts with indications and/or instructions for use.

### EXEMPLARY EMBODIMENTS

1) A composition comprising a chemical structure as follows:

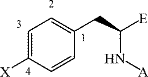

whereby A = H, Amino Acid, or Borylated Amino Acid;
E = CO₂H, B(OH)₂, CO-Amino Acid, or CO-Borylated Amino Acid, CO—NHB₁₂H₁₁; and
X = H, B(OH)₂, or B(OR)₂, or can be at position 2, 3, or 4.

2) A composition comprising a chemical structure as follows:

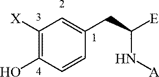

whereby A = H, Amino Acid, or Borylated Amino Acid;
E = CO₂H, B(OH)₂, CO-Amino Acid, or CO-Borylated Amino Acid, CO-NHB₁₂H_{11;} and
X = H, B(OH)₂, or B(OR)₂, or can be at position 2, or 3.

3) A composition comprising a chemical structure as follows:

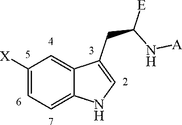

whereby A = H, Amino Acid, or Borylated Amino Acid;
E = CO₂H, B(OH)₂, CO-Amino Acid, or CO-Borylated Amino Acid, CO—NHB₁₂H₁₁; and
X = H, B(OH)₂, or B(OR)₂, or can be at position 2, 4, 5, 6, or 7.

4) A kit comprising the composition of claim 1.

5) A kit comprising the composition of claim 2.

6) A kit comprising the composition of claim 3.

7) A method of producing a composition of claim 1.

8) A method of producing a composition of claim 2.

9) A method of producing a composition of claim 3.

10) A Dosage Unit form comprising a composition of claim 1.

11) A Dosage Unit form comprising a composition of claim 2.

12) A Dosage Unit form comprising a composition of claim 3.

13) A composition comprising a chemical structure as follows:

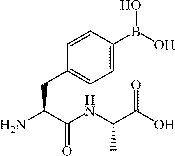 [SMILES: C[C@H](NC(=O)[C@@H](N)Cc1ccc(B(O)O)cc1)C(=O)O]

14) A composition comprising either of a chemical structure as follows:

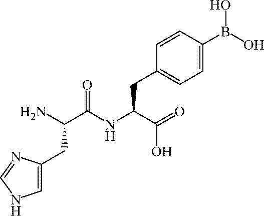 [SMILES: N[C@@H](Cc1c[nH]cn1)C(=O)N[C@@H](Cc1ccc(B(O)O)cc1)C(=O)O]

15) A composition comprising either of a chemical structure as follows:

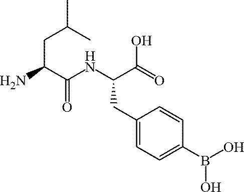 [SMILES: CC(C)C[C@H](N)C(=O)N[C@@H](Cc1ccc(B(O)O)cc1)C(=O)O]

16) A composition comprising either of a chemical structure as follows:

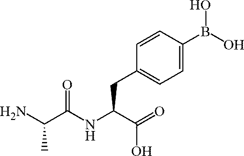 [SMILES: C[C@H](N)C(=O)N[C@@H](Cc1ccc(B(O)O)cc1)C(=O)O]

17) A composition comprising either of a chemical structure as follows:

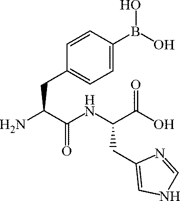 [SMILES: N[C@@H](Cc1ccc(B(O)O)cc1)C(=O)N[C@@H](Cc1c[nH]cn1)C(=O)O]

18) A composition comprising either of a chemical structure as follows:

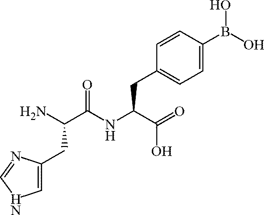 [SMILES: N[C@@H](Cc1c[nH]cn1)C(=O)N[C@@H](Cc1ccc(B(O)O)cc1)C(=O)O]

19) A composition comprising either of a chemical structure as follows:

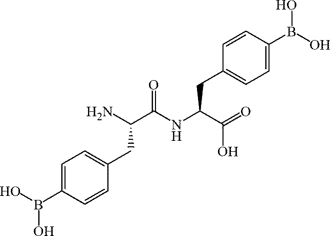 [SMILES: N[C@@H](Cc1ccc(B(O)O)cc1)C(=O)N[C@@H](Cc1ccc(B(O)O)cc1)C(=O)O]

20) A kit comprising either of the composition(s) of claim 13.

21) A kit comprising either of the composition(s) of claim 14.

22) A kit comprising either of the composition(s) of claim 15.

23) A kit comprising either of the composition(s) of claim 16.

24) A kit comprising either of the composition(s) of claim 17.

25) A kit comprising either of the composition(s) of claim 18.

26) A kit comprising either of the composition(s) of claim 19.

27) A method of producing either of a composition of claim 13.

28) A method of producing either of a composition of claim 14.

29) A method of producing either of a composition of claim 15.

30) A method of producing either of a composition of claim 16.

31) A method of producing either of a composition of claim 17.

32) A method of producing either of a composition of claim 18.

33) A method of producing either of a composition of claim 19.

34) A Dosage Unit form comprising either of a composition of claim 13.

35) A Dosage Unit form comprising either of a composition of claim 14.

36) A Dosage Unit form comprising either of a composition of claim 15.

37) A Dosage Unit form comprising either of a composition of claim 16.

38) A Dosage Unit form comprising either of a composition of claim 17.

39) A Dosage Unit form comprising either of a composition of claim 18.

40) A Dosage Unit form comprising either of a composition of claim 19.

41) A composition comprising either of a chemical structure as follows:

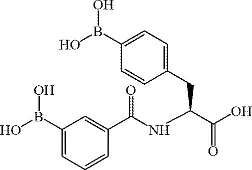 [SMILES: O=C(N[C@@H](Cc1ccc(B(O)O)cc1)C(=O)O)c1cccc(B(O)O)c1]

42) A composition comprising either of a chemical structure as follows:

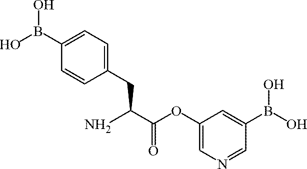 [SMILES: N[C@@H](Cc1ccc(B(O)O)cc1)C(=O)Oc1cncc(B(O)O)c1]

43) A composition comprising either of a chemical structure as follows:

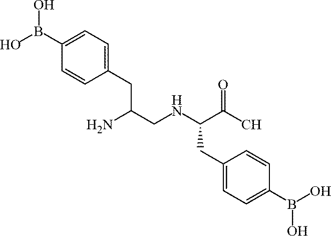 [SMILES: CC(=O)[C@H](Cc1ccc(B(O)O)cc1)NCC(N)Cc1ccc(B(O)O)cc1]

44) A kit comprising either of the composition(s) of claim 41.

45) A kit comprising either of the composition(s) of claim 42.

46) A kit comprising either of the composition(s) of claim 43.

47) A method of producing either of a composition of claim 41.

48) A method of producing either of a composition of claim 42.

49) A method of producing either of a composition of claim 43.

50) A Dosage Unit form comprising either of a composition of claim 41.

51) A Dosage Unit form comprising either of a composition of claim 42.

52) A Dosage Unit form comprising either of a composition of claim 43.

53) A method of performing Neutron Capture Therapy in the treatment of human cancer comprising:
a. synthesizing a Human Unit Dose of a borylated di-peptide amino acid (Bdi-AA). composition;
b. injecting the Bdi-AA into a tumor, whereby the Bdi-AA accumulates into a cell; and
c. irradiating the Bdi-AA with neutrons.

54) The method of claim 53, wherein the composition is selected from the group consisting of the compositions in claim(s) 1, 2, 3, 13, 14, 15, 16, 17, 18, 19, 41, 42, and 43.

55) The method of claim 53, wherein the Neutron Capture Therapy is Boron Neutron Capture Therapy.

56) The method of claim 53, wherein the irradiation comprises epithermal neutrons.

57) A method of performing Proton Boron Fusion Therapy in the treatment of human cancer comprising:
a. synthesizing a Human Unit Dose of a borylated di-peptide amino acid (Bdi-AA) composition;
b. injecting the Bdi-AA into a tumor, whereby the Bdi-AA accumulates into a cell; and
c. irradiating the Bdi-AA with protons.

58) The method of claim 57, wherein the composition is selected from the group consisting of the compositions in claim(s) 1, 2, 3, 13, 14, 15, 16, 17, 18, 19, 41, 42, and 43.

59) A composition comprising a chemical structure as follows:

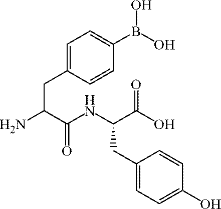 [SMILES: NC(Cc1ccc(B(O)O)cc1)C(=O)N[C@@H](Cc1ccc(O)cc1)C(=O)O]

60) A composition comprising a chemical structure as follows:

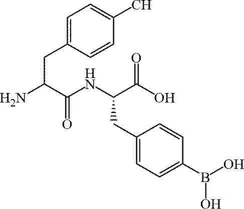 [SMILES: Cc1ccc(CC(N)C(=O)N[C@@H](Cc2ccc(B(O)O)cc2)C(=O)O)cc1]

61) A composition comprising a chemical structure as follows:

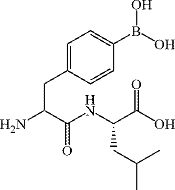 [SMILES: CC(C)C[C@H](NC(=O)C(N)Cc1ccc(B(O)O)cc1)C(=O)O]

62) A kit comprising either of the composition(s) of claim 59.

63) A kit comprising either of the composition(s) of claim 60.

64) A kit comprising either of the composition(s) of claim 61.

65) A method of producing either of a composition of claim 59.

66) A method of producing either of a composition of claim 60.

67) A method of producing either of a composition of claim 61.

68) A Dosage Unit form comprising either of a composition of claim 59.

69) A Dosage Unit form comprising either of a composition of claim 60.

70) A Dosage Unit form comprising either of a composition of claim 61.

71) A method of performing Neutron Capture Therapy in the treatment of human cancer comprising:
a. synthesizing a Human Unit Dose of a borylated di-peptide amino acid (Bdi-AA) composition;
b. injecting the Bdi-AA into a tumor, whereby the Bdi-AA accumulates into a cell; and
c. irradiating the Bdi-AA with neutrons.

72) The method of claim 71, wherein the composition is selected from the group consisting of the compositions in claim(s) 59, 60, and 61.

73) The method of claim 71, wherein the Neutron Capture Therapy is Boron Neutron Capture Therapy.

74) The method of claim 71, wherein the irradiation comprises epithermal neutrons.

75) A method of performing Proton Boron Fusion Therapy in the treatment of human cancer comprising:
a. synthesizing a Human Unit Dose of a borylated di-peptide amino acid (Bdi-AA) composition;
b. injecting the Bdi-AA into a tumor, whereby the Bdi-AA accumulates into a cell; and
c. irradiating the Bdi-AA with protons.

76) The method of claim 75, wherein the composition is selected from the group consisting of the compositions in claim(s) 59, 60, and 61.

77) A method of performing Neutron Capture Therapy in the treatment of human cancer comprising:
a. synthesizing a Human Unit Dose of a di-peptide amino acid (di-AA) composition;
b. injecting the di-AA into a tumor, whereby the di-AA accumulates into a cell; and
c. irradiating the di-AA with neutrons.

78) The method of claim 7, wherein the composition is selected from the group consisting of the compositions in claim(s) 41, 42, and 43.

79) The method of claim 77, wherein the Neutron Capture Therapy is Boron Neutron Capture Therapy.

80) The method of claim 77, wherein the irradiation comprises epithermal neutrons.

81) A method of performing Proton Boron Fusion Therapy in the treatment of human cancer comprising:
a. synthesizing a Human Unit Dose of a di-peptide amino acid (di-AA) composition;
b. injecting the di-AA into a tumor, whereby the di-AA accumulates into a cell; and
c. irradiating the di-AA with protons.

82) The method of claim 81, wherein the composition is selected from the group consisting of the compositions in claim(s) 41, 42, and 43.

### EXAMPLES

Various aspects of the invention are further described and illustrated by way of the several examples that follow, none of which is intended to limit the scope of the invention.

### Example 1: Synthesis of BPA-Ala

BPA-Ala was synthesized in the following manner. Briefly, to a solution of 20 mL dioxane and 20 mL of water at room temperature is added 2 mL of triethylamine and 2 g of 4-borono-L-phenylalanine. After fifteen (15) minutes of stirring, di-tert-butyl dicarbonate (2.3 g) is added to the reaction. After twelve (12) hrs. the reaction is complete as observed by LCMS. The dioxane is removed under reduced pressure and the aqueous is poured into 50 mL of water, and the aqueous is washed three times with 50 mL aliquots of ethyl acetate. The aqueous is then acidified with 1 M HCl, and then back extracted with 50 mL of ethyl acetate twice. The combined organic layers are washed with 50 mL of brine, then dried over magnesium sulfate. The solution is filtered, and the solvent removed under reduced pressure to reveal the following target material (2):

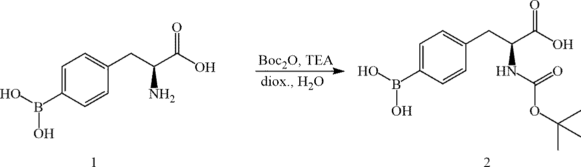

Then, to a solution of 13.4 mL dimethylformamide at room temperature is added 3.5 mL of N,N-diisopropylethylamine (DIPEA), 2 grams of Boc-BPA, 0.99 g of DL-alanine methyl ester (Ala-OMe), 1.2 g of hydroxybenzotriazole (HOBt), and 1.49 g of 1-ethyl-3-(3-dimethylaminopropyl)carbodiimide (EDC). After stirring for twelve (12) hrs., the reaction is complete as observed by LCMS. The reaction solution is poured into 50 mL of 1 M HCl,and the aqueous is washed twice with 50 mL aliquots of ethyl acetate. The combined organic layers are washed with 50 mL of brine, then dried over magnesium sulfate. The solution is filtered, and the solvent removed under reduced pressure to reveal the following target material (4):

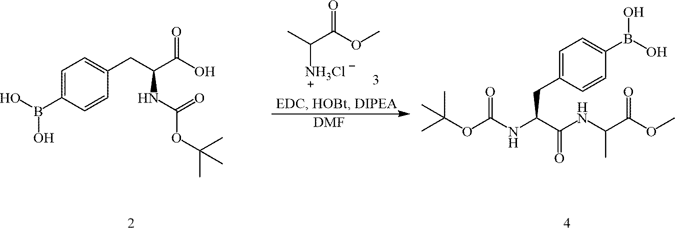

Then, to a solution of 54 mL of methanol and 6 mL of water is added 2.5 g of Boc-BPA-Ala-OMe along with 0.227 g of lithium hydroxide. After four (4) hrs. of refluxing at 65° C., the reaction is complete as observed by LCMS. The methanol was then removed under reduced pressure and the remaining aqueous layer acidified with 20 mL of 1 M HCl solution. The aqueous is then back extracted three (3) separate times with 30 mL of ethyl acetate. The combined organic layers are washed with 50 mL of brine, then dried over magnesium sulfate. The solution is then filtered, and the solvent removed under reduced pressure to reveal the following target material (5):

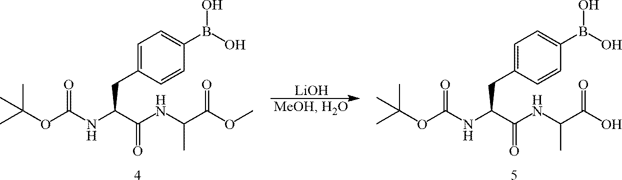

Finally, to a flask containing 2.4 g of Boc-BPA-Ala at room temperature is added 5 mL of water. Then, 23.7 mL of 4 M hydrochloric acid in 1,4-dioxane is added slowly. After two (2) hours of stirring the reaction is complete as observed by LCMS. The solvent is then removed under reduced pressure and placed onto a preparative HPLC to afford the following target material (6).

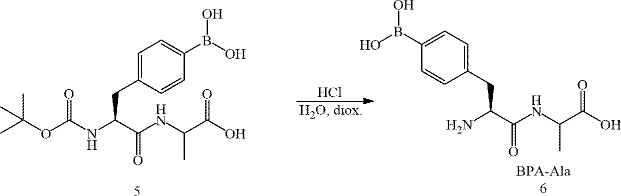

The resulting synthesis and composition denoted BPA-Ala is set forth in FIG. 1.

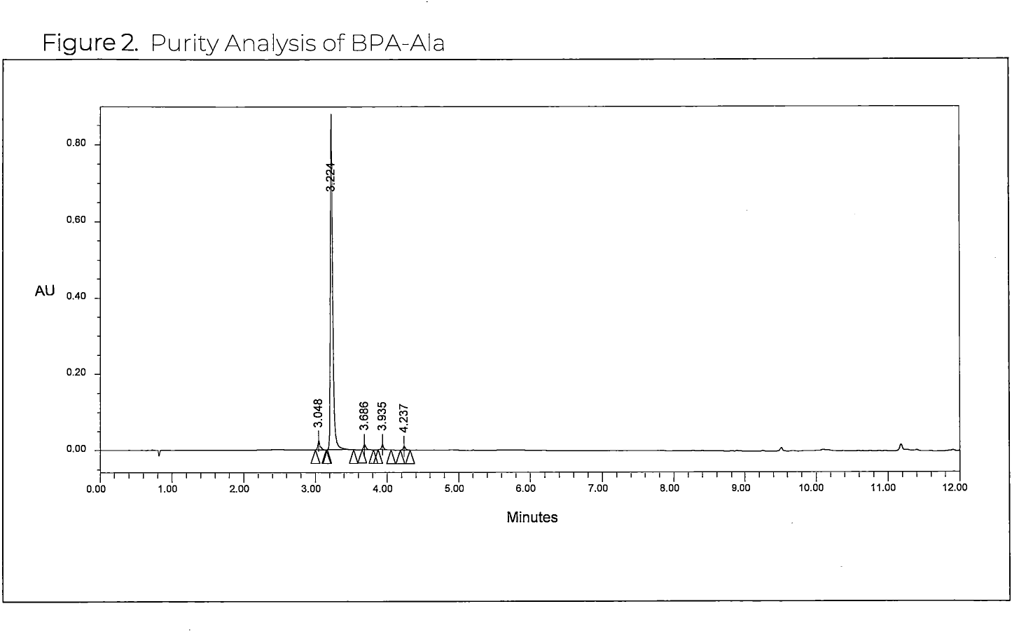

BPA-Ala was analyzed by LC/MS to confirm the molecular weight and the purity was determined after the preparation of aqueous solution. Briefly, a dipeptide was analyzed using Luna Omega Polar C18 column (2.1 × 150 mm, Phenomenex) maintained at 40° C. and with the flow rate of 0.5 ml/min. The bound test article was eluted in the gradient of acetonitrile-0.1 % TFA. The resulting purity analysis of BPA-Ala is shown in FIG. 2.

### Example 3: Synthesis of His-BPA

His-BPA was synthesized in the following manner. Briefly, to a solution of 10 mL methanol at 0° C. is added 1 g of 4-borono-L-phenylalanine. Then 0.625 mL of thionyl chloride was added dropwise. After 2 hr., the reaction is complete as observed by LCMS. The solvent is removed under reduced pressure to afford the following target material (7):

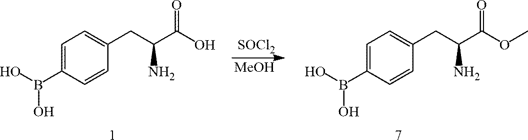

Then, to a solution of 10 mL acetonitrile at 0° C. is added 0.6 g of 4-borono-L-phenylalanine methyl ester(BPA-OMe) and 0.4 mL of triethylamine (TEA). After ten (10) minutes of stirring, 1.34 g of Boc-L-histidine(Boc)-succinimide ester (Boc-His(Boc)-OSu) is added. After stirring for twelve (12) hr., the reaction is complete as observed by LCMS. The acetonitrile is removed under reduced pressure to afford the following target material (9):

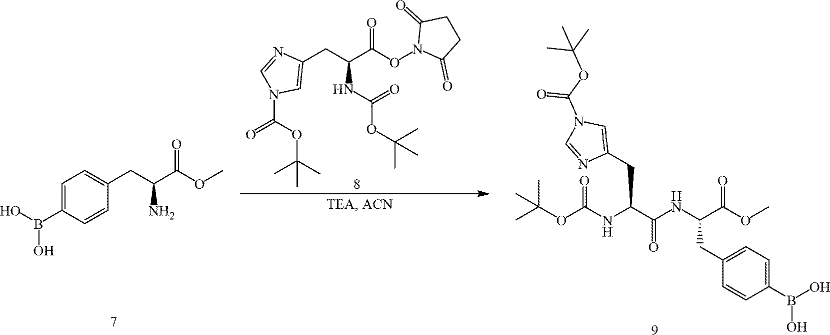

Then, to a solution of both 3.4 mL of water and 13.6 mL of methanol is 0.11 g of lithium hydroxide and 1.7 g of Boc-His(Boc)-BPA-OMe added. The reaction temperature is then brought up to 65° C. and allowed to reflux. After four (4) hr. of stirring, the reaction is complete as observed by LCMS. The solvent is removed under reduced pressure to afford the following target material (10):

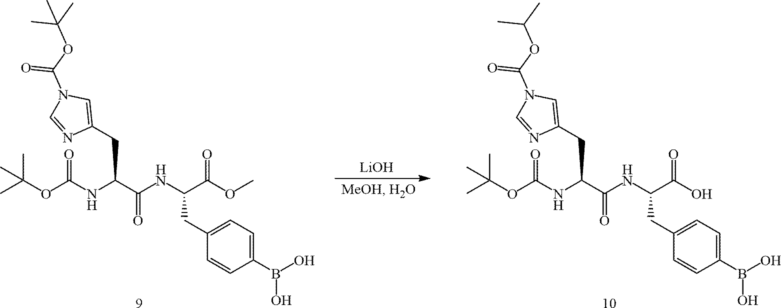

Finally, to a flask containing 1.5 g of Boc-His-BPA at room temperature is added 4.5 mL of water. Then, 10.3 mL of 4 M hydrochloric acid in 1,4-dioxane is added slowly. After two (2) hours of stirring the reaction is complete as observed by LCMS. The solvent is then removed under reduced pressure and placed onto a preparative HPLC to afford the following target material (11):

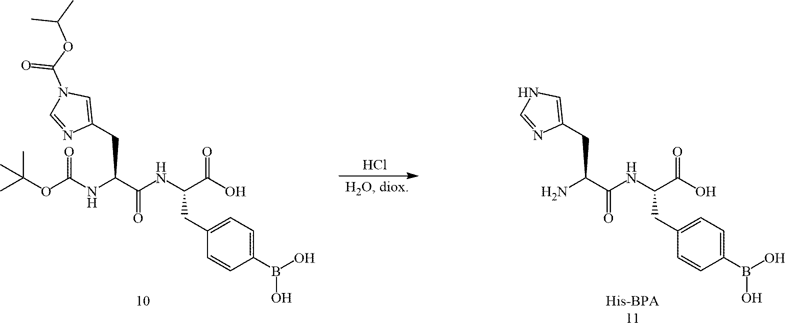

The resulting synthesis and composition denoted His-BPA is set forth in FIG. 3.

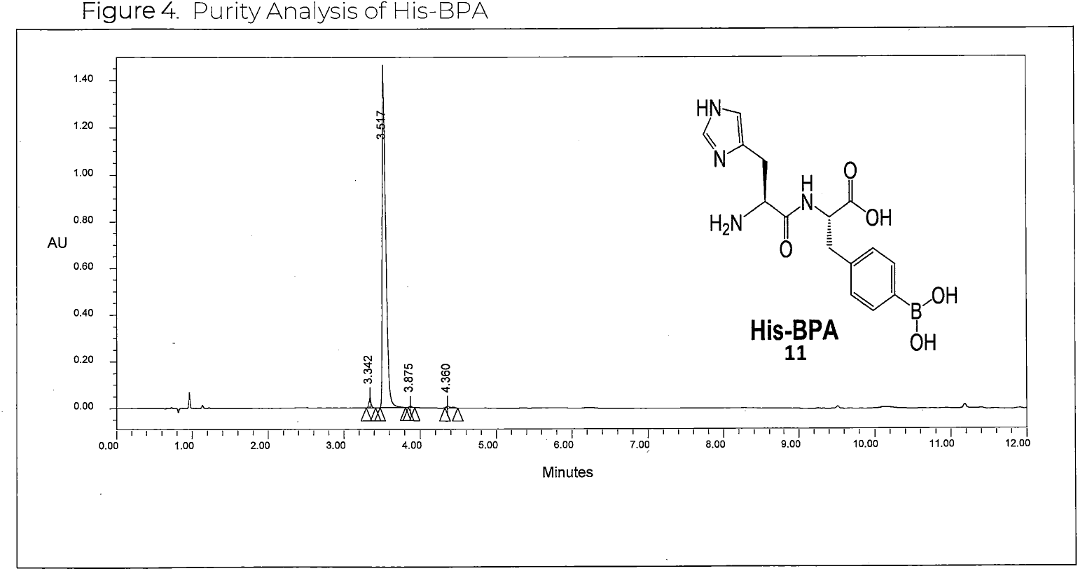

His-BPA was analyzed by LC/MS to confirm the molecular weight and the purity was determined after the preparation of aqueous solution. Briefly, a dipeptide was analyzed using Luna Omega Polar C18 column (2.1 × 150 mm, Phenomenex) maintained at 40° C. and with the flow rate of 0.5 ml/min. The bound test article was eluted in the gradient of acetonitrile-0.1% TFA. The resulting purity analysis of His-BPA is shown in FIG. 4.

### Example 3: Synthesis of Leu-BPA

Leu-BPA was synthesized in the manner. Briefly, to a solution of 10 mL of water and 2.5 mL of dioxane at 20° C. is added 0.64 g of sodium hydroxide and 1 g of L-leucine. After 15 minutes of stirring, 2.5 g of di-tert-butyl dicarbonate is added to the reaction. After four (4) hrs., the reaction is complete as observed by LCMS. The dioxane is removed under reduced pressure and an additional 10 mL of water was added to the aqueous. The aqueous is then acidified with 1 M hydrochloric acid, and then back extracted with 30 mL of ethyl acetate 3 times. The combined organic fractions were washed with 50 mL of brine then dried over magnesium sulfate. The solution was then filtered, and the solvent removed under reduced pressure to afford the following target material (13):

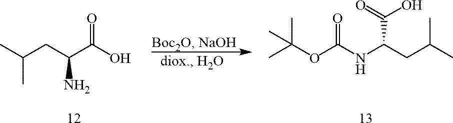

Then, to a solution of 38 mL of methylene chloride at room temperature is added 1.76 g Boc-Leu, 1.365 g of N-hydroxyphthalimide (NHP), 0.093 g of 4-dimethylaminopyridine (DMAP), and 1.311 mL of diisopropylcarbodiimide (DIC). After 12 hr., the reaction is complete as observed by LCMS. The solvent is removed under reduced pressure and the concentrate dissolved in 30 mL of water. The aqueous is then extracted 3 times with 30 mL of ethyl acetate. The combined organic layers are washed with 50 mL of brine, then dried over magnesium sulfate. After filtering, the solvent is removed under reduced pressure and subjected to flash column chromatography. After combining the fractions, the solvent is removed under reduced pressure to reveal the following target material (14):

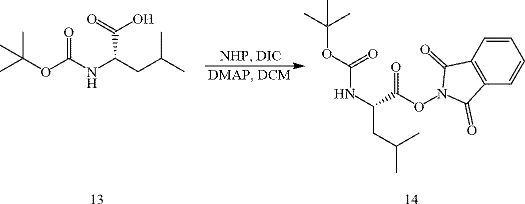

Then, 4-borono-L-phenylalanine methyl ester (BPA-OMe) was synthesized in the following manner. Briefly, to a solution of 10 mL methanol at 0° C. is added 1 g of 4-borono-L-phenylalanine. Then 0.625 mL of thionyl chloride was added in dropwise. After 2 hr., the reaction is complete as observed by LCMS. The solvent is removed under reduced pressure to afford the following target material (7):

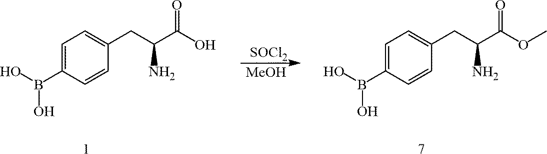

Then, to a solution of 10 mL of acetonitrile at room temperature is added 0.593 g of BPA-OMe and 0.44 mL of triethylamine. After fifteen (15) minutes of stirring, 1 g of Boc-L-leucine O-phthalimide ester is added. After four (4) hrs., the reaction is complete as observed by LCMS. The solvent is removed under reduced pressure to reveal the following target material (15):

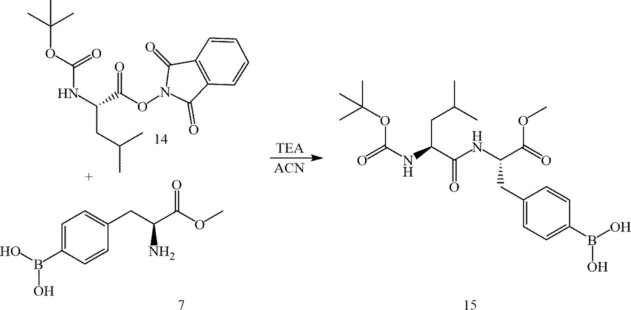

Then, to a solution of 24 mL of methanol and 6.5 mL of water is added 1.2 g of Boc-Leu-BPA-OMe along with 0.198 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three separate times with 30 mL of ethyl acetate. The combined organic layers are washed with 50 mL of brine, then dried over magnesium sulfate. The solution is then filtered, and the solvent removed under reduced pressure to reveal the following target material (16):

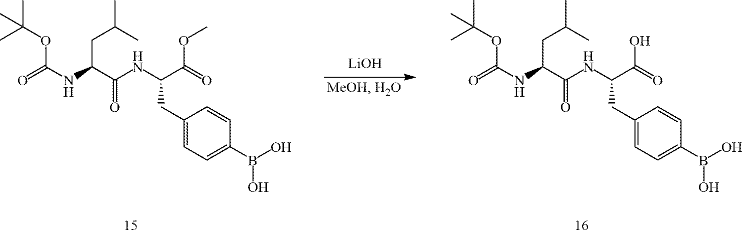

Finally, to a flask containing 1.2 g of Boc-Leu-BPA at room temperature is added 3 mL of water. Then, 14.2 mL of 4 M hydrochloric acid in 1,4-dioxane is added slowly. After 2 hours of stirring the reaction is complete as observed by LCMS. The solvent is then removed under reduced pressure and placed onto a preparative HPLC to afford the following target material (17):

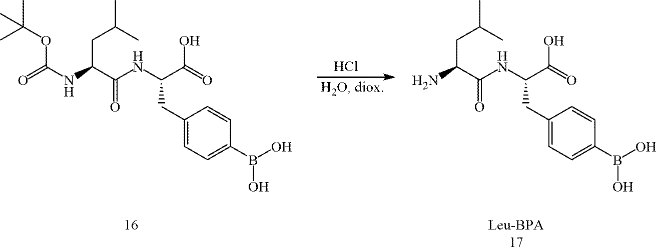

The resulting synthesis and composition denoted Leu-BPA is set forth in FIG. 5.

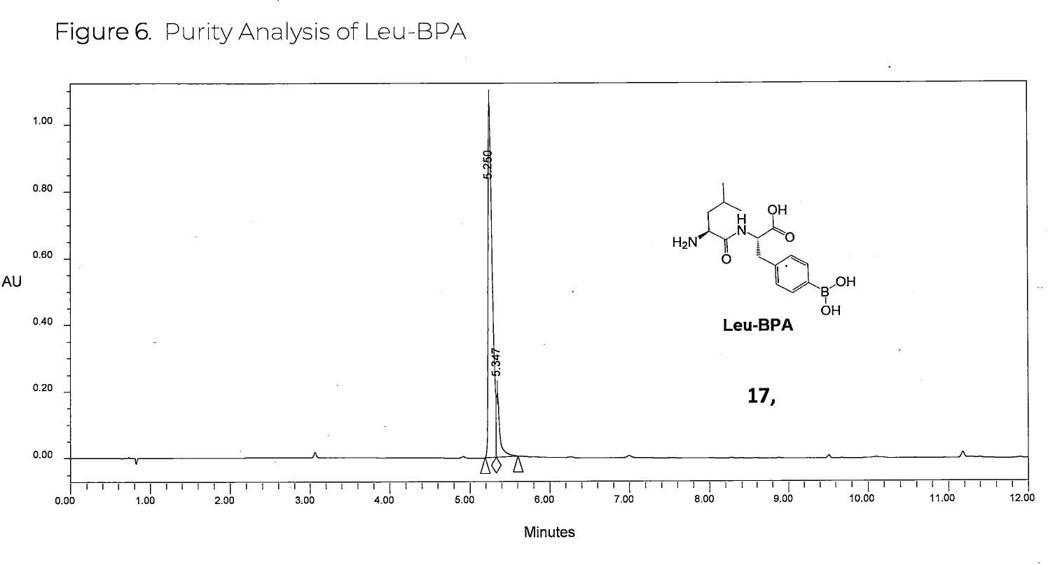

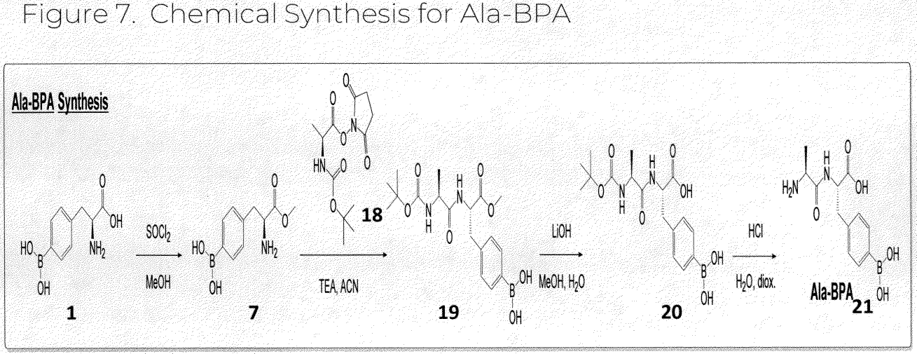

Leu-BPA was analyzed by LC/MS to confirm the molecular weight and the purity was determined after the preparation of aqueous solution. Briefly, a dipeptide was analyzed using Luna Omega Polar C18 column (2.1 × 150 mm, Phenomenex) maintained at 40° C. and with the flow rate of 0.5 ml/min. The bound test article was eluted in the gradient of acetonitrile-0.1% TFA. The resulting purity analysis of Leu-BPA is shown in FIG. 6.

### Example 4: Synthesis of Ala-BPA

Ala-BPA was synthesized in the following manner. Briefly, to a solution of ten (10) mL methanol at 0° C. is added 1 g of 4-L-Boronophenylalanine. Then 0.625 mL of thionyl chloride was added in dropwise. After 2 hr., the reaction is complete as observed by LCMS. The solvent is removed under reduced pressure to afford the following target material (7):

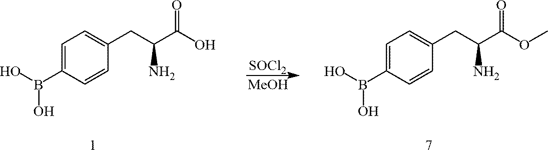

Then, to a flask containing 28 mL of acetonitrile at room temperature is added 1.5 g of BPA-OMe and 1.125 mL of triethylamine. After 10 minutes of stirring, 1.925 g of Boc-L-Ala-succinimide ester is added. After 4 hr., the reaction is complete as observed by LCMS. The solvent is removed under reduced pressure to reveal the following target material (19):

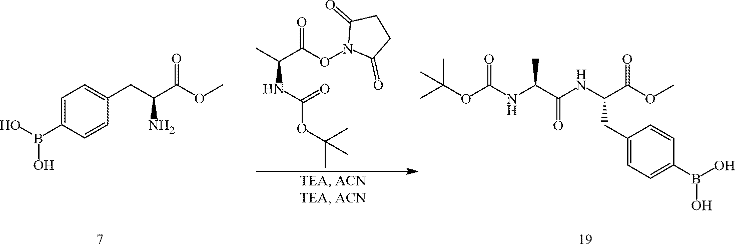

Then, to a solution of 52 mL of methanol and 6.5 mL of water is added 2.6 g of Boc-Ala-BPA-OMe along with 0.237 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three separate times with 30 mL of ethyl acetate. The combined organic layers are washed with 50 mL of brine, then dried over magnesium sulfate. The solution is then filtered, and the solvent removed under reduced pressure to reveal the following target material (20):

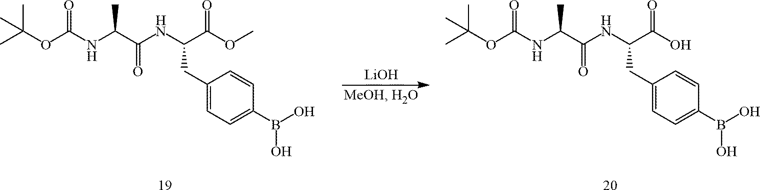

Finally, to a flask containing 2.5 g of Boc-Ala-BPA at room temperature is added 5 mL of water. Then, 16.4 mL of 4 M hydrochloric acid in 1,4-dioxane is added slowly. After 2 hours of stirring the reaction is complete as observed by LCMS. The solvent is then removed under reduced pressure and placed onto a preparative HPLC to afford the following target material (21):

The resulting synthesis and composition denoted Ala-BPA is set forth in FIG. 7. Ala-BPA was analyzed by LC/MS to confirm the molecular weight and the purity was determined after the preparation of aqueous solution. A dipeptide was analyzed using Acquity BEH C18 column (2.1 × 50 mm, Waters) maintained at 40° C. and with the flow rate of 0.5 ml/min. The bound test article was eluted in the gradient of acetonitrile-0.1% formic acid. The resulting purity analysis of Ala-BPA is shown in FIG. 8.

### Example 5: Synthesis of BPA-BPA

BPA-BPA was synthesized in the following manner. Briefly, to a solution of 20 mL dioxane and 20 mL of water at room temperature is added 2 mL of triethylamine and 2 g of 4-borono-L-phenylalanine (BPA). After 15 minutes of stirring, di-tert-butyl dicarbonate (2.3 g) is added to the reaction. After 12 hr. the reaction is complete as observed by LCMS. The dioxane is removed under reduced pressure and the aqueous is poured into 50 mL of water, and the aqueous is washed three times with 50 mL aliquots of ethyl acetate. The aqueous is then acidified with 1 M HCl,and then back extracted with 50 mL of ethyl acetate twice. The combined organic layers are washed with 50 mL of brine, then dried over magnesium sulfate. The solution is filtered, and the solvent removed under reduced pressure to reveal the following target material (2):

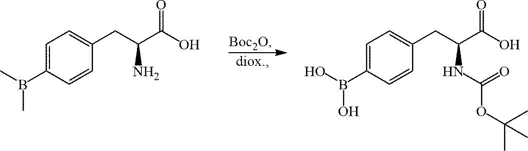

Then, to a solution 1 of 10 mL methanol at 0° C. is added 1 g of 4-borono-L-phenylalanine. Then, 0.625 mL of thionyl d in dropwise. After 2 hr., the reaction is complete as observed by LCMS. The solve er reduced pressure to afford the following target material (7):

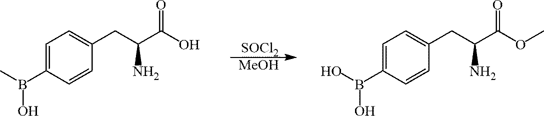 [SMILES: "MeOH" ; CB(O)c1ccc(C[C@H](N)C(=O)O)cc1 ; "SOCl2" ; COC(=O)[C@@H](N)Cc1ccc(B(O)O)cc1]

Then, to a solution of 15 mL dimethylformamide at room temperature is added 2.4 mL of triethylamine (TEA), 1.89 g of Boc-BPA (2), 1.24 g of BPA-OMe (7), 1.01 g of hydroxybenzotriazole (HOBt), and 1.27 g of 1-ethyl-3-(3-dimethylaminopropyl)carbodiimide (EDC). After stirring for twelve (12) hrs., the reaction is complete as observed by LCMS. The reaction solution is poured into 50 mL of 1 M HCl,and the aqueous is washed twice with 50 mL aliquots of ethyl acetate. The combined organic layers are washed with 50 mL of brine, then dried over magnesium sulfate. The solution is filtered, and the solvent removed under reduced pressure to reveal the following target material (22):

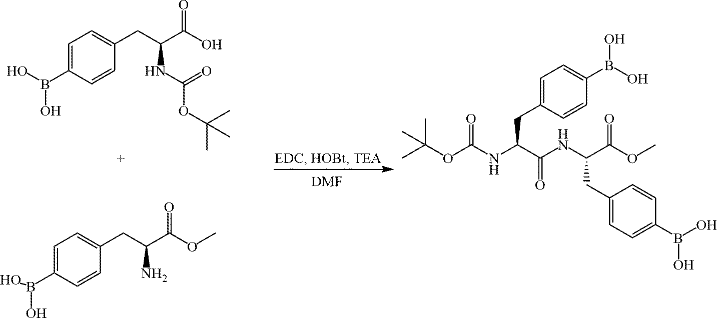

Then, to a solution of 36 mL of methanol and 4 mL of water is added 2.2 g of Boc-BPA-BPA-OMe (22) along with 0.151 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three times with 30 mL of ethyl acetate. The combined organic layers are washed with 50 mL of brine, then dried over magnesium sulfate. The solution is then filtered, and the solvent removed under reduced pressure to reveal the following target material (23):

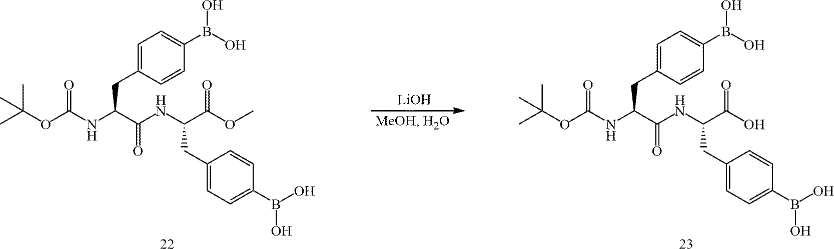

Finally, to a flask containing 1.1 g of Boc-BPA-BPA (23) at room temperature is added 2.6 mL of water. Then, 10.6 mL of 4 M hydrochloric acid in 1,4-dioxane is added slowly. After two (2) hours of stirring the reaction is complete as observed by LCMS. The solvent is then removed under reduced pressure and placed onto a preparative HPLC to afford the following target material (24):

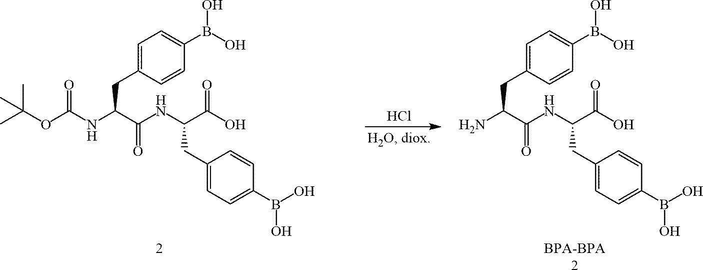

The resulting synthesis and composition denoted BPA-BPA is set forth in FIG. 9.

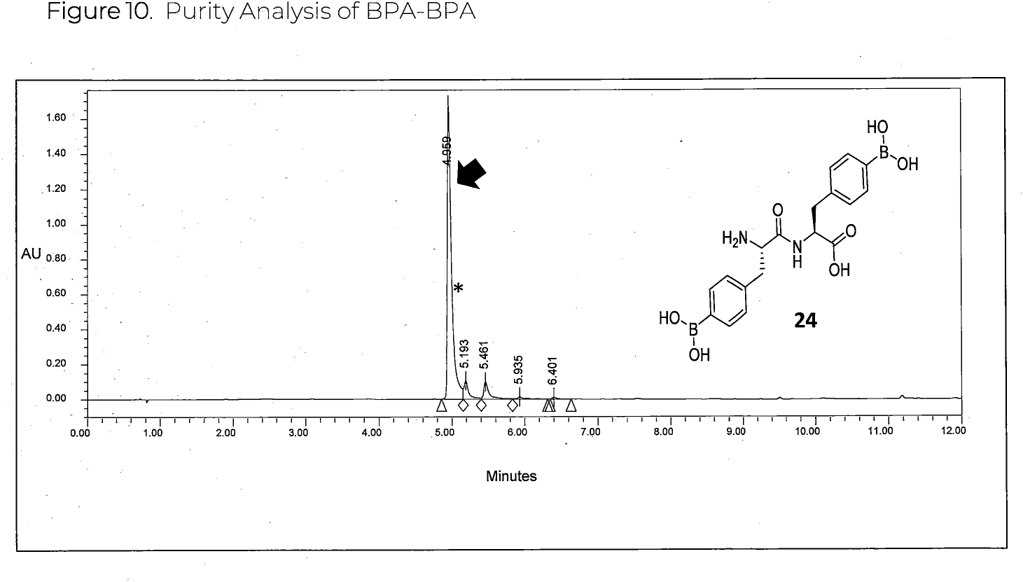

BPA-BPA was analyzed by LC/MS to confirm the molecular weight and the purity was determined after the preparation of aqueous solution. Briefly, a dipeptide was analyzed using Luna Omega Polar C18 column (2.1 × 150 mm, Phenomenex) maintained at 40° C. and with the flow rate of 0.5 ml/min. The bound test article was eluted in the gradient of acetonitrile-0.1% TFA. The resulting purity analysis of BPA-BPA is shown in FIG. 10.

### Example 6: Synthesis of 3-boronobenzyl-BPA

3-Boronobenzyl-BPA was synthesized in the following manner. Briefly, to a solution of 15 mL dimethylformamide at room temperature is added 1.7 mL of diisoprpylethylamine and 1.0 g of 4-boron-L-phenylalanine methyl ester (7). Then 3-boronobenzoic acid (25, 0.82 g) is added to the reaction. The coupling agents EDC (1.0 g) and HOBt (0.82 g) are then added to the reaction. After 12 hr., the reaction is complete as observed by LCMS. The reaction solution is poured into 100 mL of 1 M HCl and 100 mL of ethyl acetate, the organic layer is separated. The organic layer is washed with 50 mL of saturated sodium bicarbonate, and then 50 mL of brine, then dried over magnesium sulfate. The solution is filtered, and the solvent removed under reduced pressure to reveal the following target material (26):

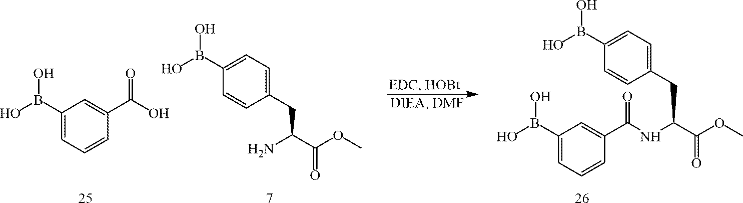

Then, to a flask containing 3-boronobenzyl-BPA-OMe (26) dissolve in 12 mL MeOH and 3 mL water, at room temperature is added 0.15 g of LiOH. The reaction was brought to 60° C. After 2 hours the reaction is complete as observed by LCMS. The material was purified by prep-LC to reveal the following target material (27).

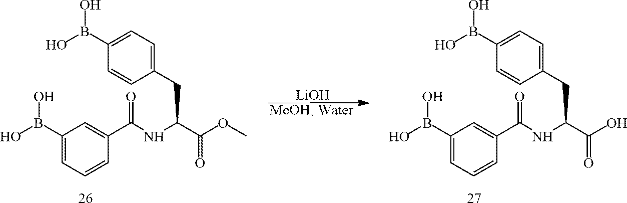

The resulting synthesis and composition denoted 3-boronobenzyl-BPA is set forth in FIG. 11.

### Example 7: Synthesis of BPA-3-boronopyridine

BPAboronopyridine was synthesized in the following manner. Briefly, to a solution of 15 mL dimethylformamide at room temperature is added 0.62 mL of diisoprpylethylamine and 0.5 g of Boc-4-boron-L-phenylalanine (2). Then 3-boronopyridine (28, 0.36 g) is added to the reaction. The coupling agents EDC (0.37 g) and HOBt (0.3 g) are then added to the reaction. After 12 hrs., the reaction is complete as observed by LCMS. The reaction solution is then poured into 100 mL of 1 M HCl and 100 mL of ethyl acetate, the organic layer is separated. The organic layer is washed with 50 mL of saturated sodium bicarbonate, and then 50 mL of brine, then dried over magnesium sulfate. The solution is filtered, and the solvent removed under reduced pressure to reveal the target material (29).

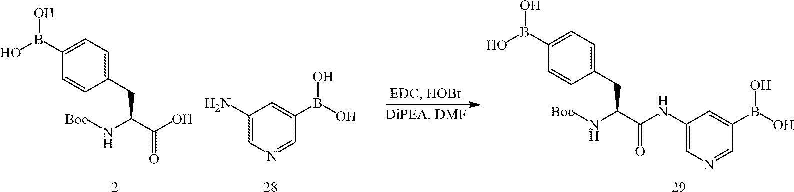

Then, to a flask containing Boc-BPA-3-boronopyridine at room temperature is added 6.6 mL of 4 M HCl in dioxane. After thirty (30) minutes the reaction is complete as observed by LCMS. The solvent is removed under reduced pressure to reveal the target material (30).

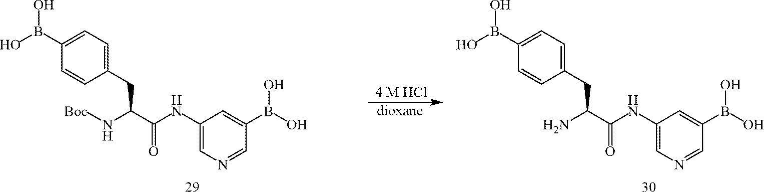

The resulting synthesis and composition denoted BPA-3-boronopyridine is set forth in FIG. 12.

### Example 8: Synthesis of Reduced BPA-BPA ((Red)-BPA-BPA)

Reduced BPA-BPA (33) was synthesized in the following manner. Briefly, to a solution of 50 mL tetrahydrofuran at 0° C. is added 3.3 g mL of 4-boron-L-phenylalanine methyl ester (7). Then 3.3 g of lithium aluminum hydride is added portion wise to the reaction. The reaction was complete at two (2) hours as observed by LCMS. Following the Fieser workup the intermediate alcohol was re-suspended in 75 mL of dioxane/water mix at room temperature. To this was added 4 mL of triethylamine and 4.5 g of di-tert-butyl dicarbonate. The boc protected BPA alcohol was purified by ethyl acetate extraction, and then dissolved in dichloromethane, and the temperature was brought down to -76° C.

To this solution was added 3 mL of triethylamine, 0.8 mL of dimethyl sulfoxide, and finally 2.5 mL of oxalyl chloride. After 20 min the reaction was extracted reveling the aldehyde of BPA (31). The BPA aldehyde was dissolved in 45 mL of methanol and v/v 5% acetic acid. To this was added 800 mg of 4-boron-L-phenylalanine methyl ester (7), and 425 mg of sodium cyanoborohydride at room temperature. After two (2) hrs. the target secondary amine was isolated via extraction into ethyl acetate.

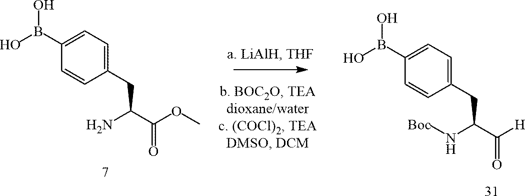

Then, to a flask containing Boc-BPA-NH-BPA-OMe (32) dissolved in 8 mL MeOH and 8 mL water, at room temperature is added 0.1 g of LiOH. The reaction was brought to 60° C. After 2 hours the reaction is complete as observed by LCMS. The material was extracted into acidic water, followed by lyophilization which removed the boc group in-situ.

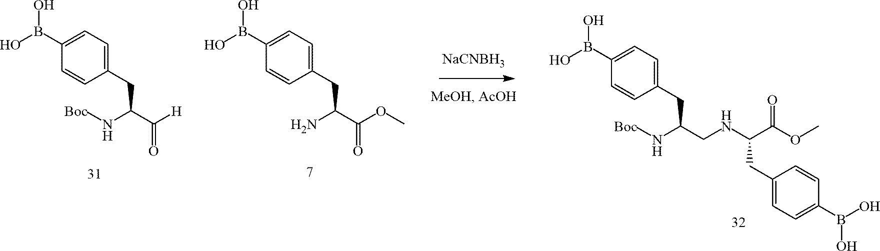

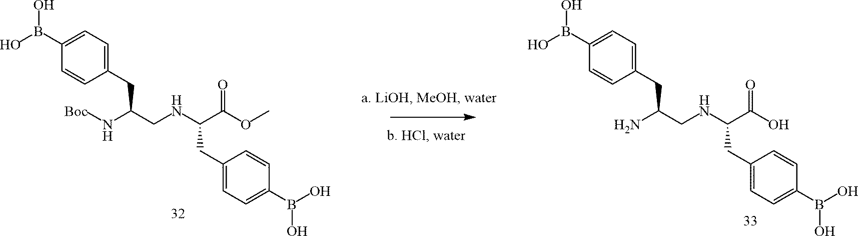

The resulting synthesis and composition denoted (Red)-BPA-BPA is set forth in FIG. 13.

### Example 9: Synthesis of BPA-Tyr

BPA-Tyr (36) was synthesized in the following manner. Briefly, to a solution of 20 mL dioxane and 20 mL of water at room temperature is added 2 mL of triethylamine and 2 g of 4-borono-L-phenylalanine. After fifteen (15) minutes of stirring, di-tert-butyl dicarbonate (2.3 g) is added to the reaction. After twelve (12) hrs. the reaction is complete as observed by LCMS. The dioxane is removed under reduced pressure and the aqueous is poured into 50 mL of water, and the aqueous is washed three times with 50 mL aliquots of ethyl acetate. The aqueous is then acidified with 1 M HCl, and then back extracted with 50 mL of ethyl acetate twice. The combined organic layers are washed with 50 mL of brine, then dried over magnesium sulfate. The solution is filtered, and the solvent removed under reduced pressure to reveal the following target material:

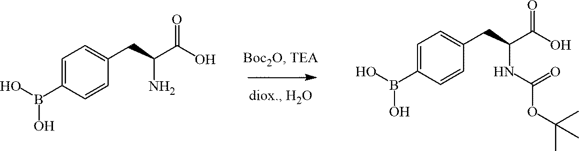

Then, to a solution of 20 mL methanol at 0° C. is added 2 g of L-tyrosine. Then 2.843 mL of thionyl chloride was added dropwise. After 3 hr., the reaction is complete as observed by LCMS. The solvent is removed under reduced pressure to afford the following target material:

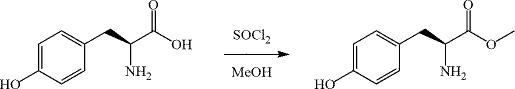

Then, to a solution of 8.5 mL dimethylformamide at room temperature is added 2.8 mL of diisopropylethylamine (DIPEA), 1.74 g of Boc-BPA, 1.0 g of Tyr-OMe, 1.04 g of hydroxybenzotriazole (HOBt), and 1.18 g of 1-ethyl-3-(3-dimethylaminopropyl)carbodiimide (EDC). After stirring for twelve (12) hrs., the reaction is complete as observed by LCMS. The reaction solution is poured into 50 mL of 1 M HCl,and the aqueous is washed twice with 50 mL aliquots of ethyl acetate. The combined organic layers are washed with 50 mL of brine, then dried over magnesium sulfate. The solution is filtered, and the solvent removed under reduced pressure to reveal the following target material:

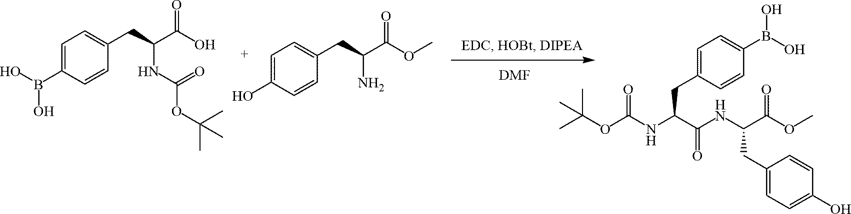

Then, to a solution of 40 mL of methanol and 2.5 mL of water is added 2.5 g of Boc-BPA-Tyr-OMe along with 0.648 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three times with 30 mL of ethyl acetate. The combined organic layers are washed with 50 mL of brine, then dried over magnesium sulfate. The solution is then filtered, and the solvent removed under reduced pressure to reveal the following target material:

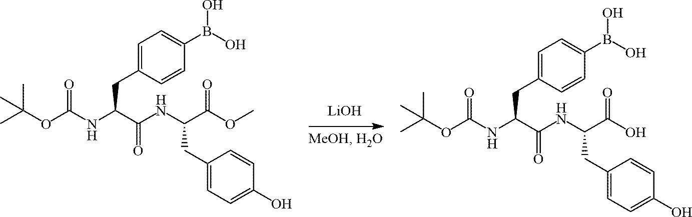

Finally, to a flask containing 2.4 g of Boc-BPA-Tyr at room temperature is added 3.0 mL of water. Then, 24.1 mL of 4 M hydrochloric acid in 1,4-dioxane is added slowly. After two (2) hours of stirring the reaction is complete as observed by LCMS. The solvent is then removed under reduced . pressure and placed onto a preparative HPLC to afford the following target material (36):

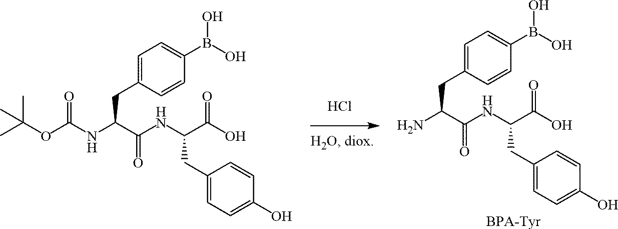

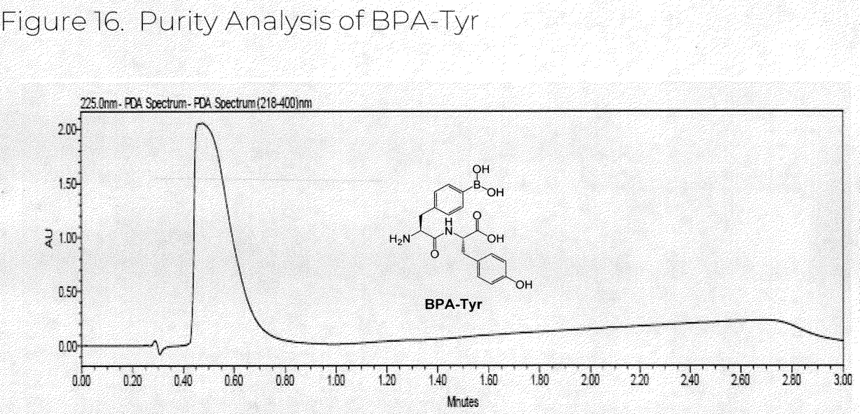

The resulting synthesis and composition denoted BPA-Tyr (36) is set forth in FIG. 15. BPA-Tyr was analyzed by LC/MS to confirm the molecular weight and the purity was determined after the preparation of aqueous solution. Briefly, a dipeptide was analyzed using BEH C18 column (2.1 × 50 mm, Waters) maintained at 40° C. and with the flow rate of 0.5 ml/min. The bound test article was eluted in the gradient of acetonitrile-0.1% FA. The resulting purity analysis of BPA-Tyr is shown in FIG. 16.

### Example 10: Synthesis of Tyr-BPA

Tyr-BPA (37) was synthesized in the following manner. Briefly, to a solution of 23 mL dioxane and 20 mL of water at room temperature is added 2.3 mL of triethylamine and 2 g of L-tyrosine. After fifteen (15) minutes of stirring, di-tert-butyl dicarbonate (2.65 g) is added to the reaction. After twelve (12) hrs. the reaction is complete as observed by LCMS. The dioxane is removed under reduced pressure and the aqueous is poured into 50 mL of water, and the aqueous is washed three times with 50 mL aliquots of ethyl acetate. The aqueous is then acidified with 1 M HCl, and then back extracted with 50 mL of ethyl acetate twice. The combined organic layers are washed with 50 mL of brine, then dried over magnesium sulfate. The solution is filtered, and the solvent removed under reduced pressure to reveal the following target material:

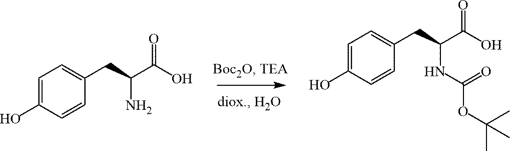

Then, to a solution of 20 mL methanol at 0° C. is added 2 g of 4-borono-L-phenylalanine. Then, 2.88 mL of thionyl chloride was added in dropwise. After 2 hr., the reaction is complete as observed by LCMS. The solvent is removed under reduced pressure to afford the following target material:

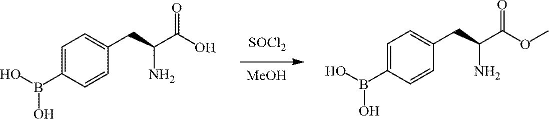 [SMILES: "MeOH" ; N[C@@H](Cc1ccc(B(O)O)cc1)C(=O)O ; "SOCl2" ; COC(=O)[C@@H](N)Cc1ccc(B(O)O)cc1]

Then, to a solution of 6.0 mL dimethylformamide at room temperature is added 1.9 mL of diisopropylethylamine (DIPEA), 1.0 g of Boc-Tyr, 0.872 g of BPA-OMe, 0.721 g of hydroxybenzotriazole (HOBt), and 0.818 g of 1-ethyl-3-(3-dimethylaminopropyl)carbodiimide (EDC). After stirring for twelve (12) hrs., the reaction is complete as observed by LCMS. The reaction solution is poured into 50 mL of 1 M HCl,and the aqueous is washed twice with 50 mL aliquots of ethyl acetate. The combined organic layers are washed with 50 mL of brine, then dried over magnesium sulfate. The solution is filtered, and the solvent removed under reduced pressure to reveal the following target material:

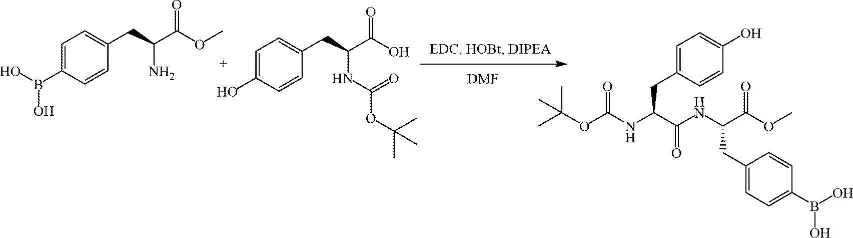

Then, to a solution of 24.1 mL of methanol and 1.5 mL of water is added 1.5 g of Boc-Tyr-BPA-OMe along with 0.222 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three times with 30 mL of ethyl acetate. The combined organic layers are washed with 50 mL of brine, then dried over magnesium sulfate. The solution is then filtered, and the solvent removed under reduced pressure to reveal the following target material:

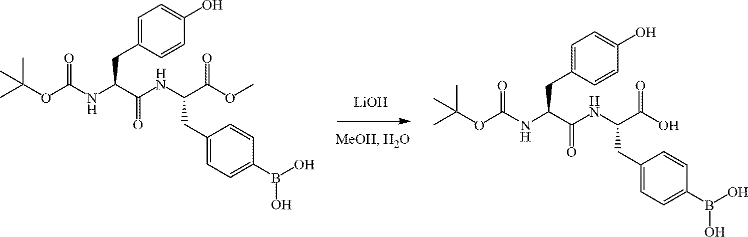

Finally, to a flask containing 1.18 g of Boc-Tyr-BPA at room temperature is added 11.8 mL of 4 M hydrochloric acid in 1,4-dioxane slowly. After two (2) hours of stirring the reaction is complete as observed by LCMS. The solvent is then removed under reduced pressure and placed onto a preparative HPLC to afford the following target material (37):

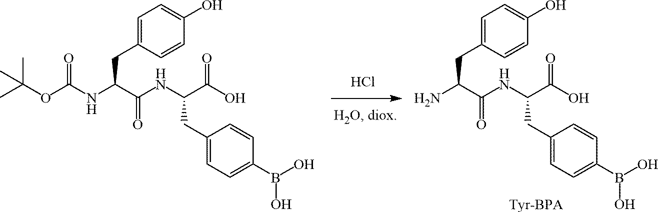

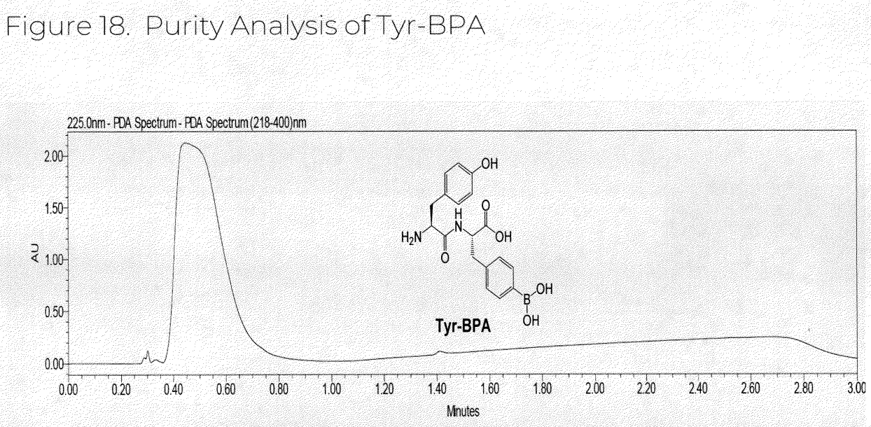

The resulting synthesis and composition denoted Tyr-BPA (37) is set forth in FIG. 17. Tyr-BPA was analyzed by LC/MS to confirm the molecular weight and the purity was determined after the preparation of aqueous solution. Briefly, a dipeptide was analyzed using BEH C18 column (2.1 × 50 mm, Waters) maintained at 40° C. and with the flow rate of 0.5 ml/min. The bound test article was eluted in the gradient of acetonitrile-0.1% FA. The resulting purity analysis of Tyr-BPA is shown in FIG. 18.

### Example 11: Synthesis of BPA-Leu

BPA-Leu (38) was synthesized in the following manner. Briefly, to a solution of 20 mL dioxane and 20 mL of water at room temperature is added 2 mL of triethylamine and 2 g of 4-borono-L-phenylalanine. After fifteen (15) minutes of stirring, di-tert-butyl dicarbonate (2.3 g) is added to the reaction. After twelve (12) hrs. the reaction is complete as observed by LCMS. The dioxane is removed under reduced pressure and the aqueous is poured into 50 mL of water, and the aqueous is washed three times with 50 mL aliquots of ethyl acetate. The aqueous is then acidified with 1 M HCl, and then back extracted with 50 mL of ethyl acetate twice. The combined organic layers are washed with 50 mL of brine, then dried over magnesium sulfate. The solution is filtered, and the solvent removed under reduced pressure to reveal the following target material:

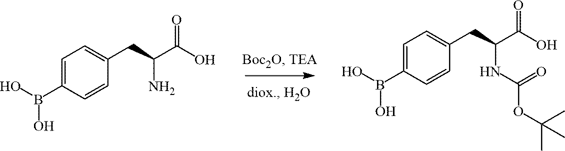

Then, to a solution of 20 mL methanol at 0° C. is added 2 g of L-leucine. Then, 2.88 mL of thionyl chloride was added in dropwise. After 2 hr., the reaction is complete as observed by LCMS. The solvent is removed under reduced pressure to afford the following target material:

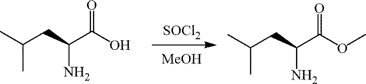

Then, to a solution of 11.5 mL dimethylformamide at room temperature is added 3.7 mL of diisopropylethylamine (DIPEA), 2.3 g of Boc-Tyr, 1.0 g of Leu-OMe, 1.39 g of hydroxybenzotriazole (HOBt), and 1.58 g of 1-ethyl-3-(3-dimethylaminopropyl)carbodiimide (EDC). After stirring for twelve (12) hrs., the reaction is complete as observed by LCMS. The reaction solution is poured into 50 mL of 1 M HCl, and the aqueous is washed twice with 50 mL aliquots of ethyl acetate. The combined organic layers are washed with 50 mL of brine, then dried over magnesium sulfate. The solution is filtered, and the solvent removed under reduced pressure to reveal the following target material:

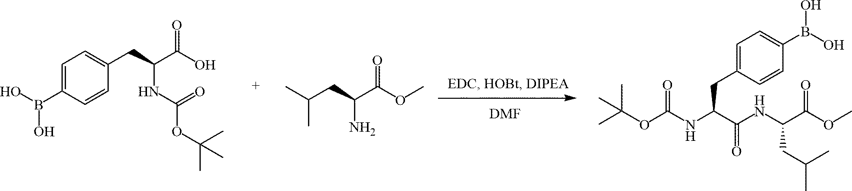

Then, to a solution of 48 mL of methanol and 3.0 mL of water is added 3.0 g of Boc-BPA-Leu-OMe along with 0.494 g of lithium hydroxide. After 4 hr. of refluxing at 65° C., the reaction is complete as observed by LCMS. The methanol was then removed under reduced pressure and the remaining aqueous layer acidified with 20 mL of 1 M HCl solution. The aqueous is then back extracted three times with 30 mL of ethyl acetate. The combined organic layers are washed with 50 mL of brine, then dried over magnesium sulfate. The solution is then filtered, and the solvent removed under reduced pressure to reveal the following target material:

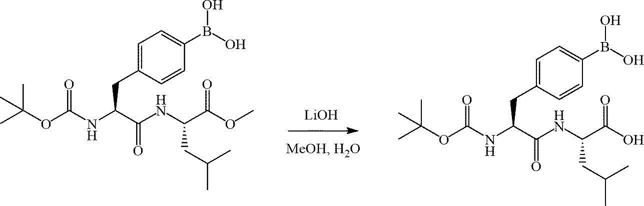

Finally, to a flask containing 2.9 g of Boc-BPA-Leu at room temperature is added 3.0 mL of water. Then, 26.1 mL of 4 M hydrochloric acid in 1,4-dioxane is added slowly. After two (2) hours of stirring the reaction is complete as observed by LCMS. The solvent is then removed under reduced pressure and placed onto a preparative HPLC to afford the following target material (38):

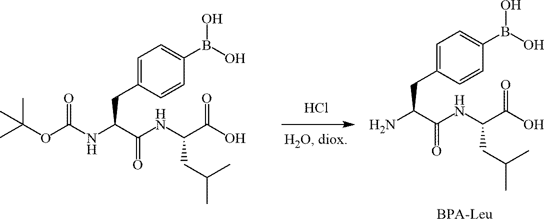

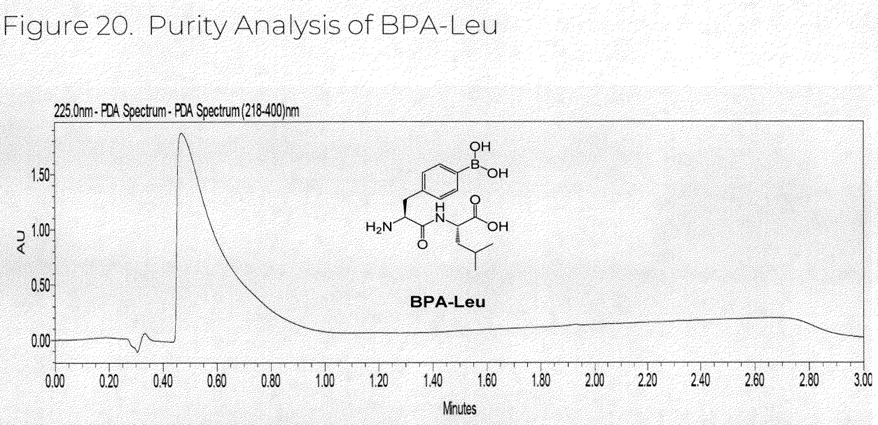

The resulting synthesis and composition denoted BPA-Leu (38) is set forth in FIG. 19. BPA-Leu was analyzed by LC/MS to confirm the molecular weight and the purity was determined after the preparation of aqueous solution. Briefly, a dipeptide was analyzed using BEH C18 column (2.1 × 50 mm, Waters) maintained at 40° C. and with the flow rate of 0.5 ml/min. The bound test article was eluted in the gradient of acetonitrile-0.1% FA. The resulting purity analysis of BPA-Leu is shown in FIG. 20.

### Example 12: Evaluation of Borylated Dipeptides Uptake in Multiple Cancer Cell Lines

In this experiment, a panel of dipeptides were evaluated in which each amino acid adjacent to BPA is a putative substrate for LAT-1. The set of amino acids was chosen from Leu, His and Ala as well as BPA with the objective being that an increased solubility in solution will overcome the drawbacks of BPA as well as will be able to take advantage of an alternative uptake mechanism and ultimately, achieve higher boron-10 concentration in tumors while maintaining a putative 3:1 tumor-to blood ratio or more. Utilization of the following materials and methods were provided by the following protocol.

The indicated cancer cell line (A) or FaDu cells (B) were harvested from culture and washed twice with PBS. Cell lines were adjusted to 2 million cells/ml in Hanks Balanced Salt Solution (HBSS). BPA or dipeptide boron compounds were added to cells at the final concentration of 2.5 mM in HBSS and the treated cells were incubated at 37° C. 5% CO2 for 2 hrs with shaking. The cells were collected by centrifugation; washed with cold PBS twice and reconstituted in 1 ml of cold PBS. Then, 50 µl of cell suspension was removed, pelleted, and lysed using RIPA buffer and the protein concentration was determined using BCA assay. The remaining 950 µl was used for boron compounds uptake measurements using ICP-OES.

To this end, 0.5 mL of concentrated nitric acid was added to each cell pellet in a 15 mL conical tube. The tubes were capped and placed in an oven set at 80° C. for 3 hours. 2 mL of water was added’ to each sample to bring the final volume up to 2.5 mL. The boron content in each sample was then measured on an Agilent 5110 ICP-OES using an Agilent SPS4 Autosampler for sample introduction.

The data was analyzed using Agilent’s ICP Expert Software, version 7.4.2.10790. Boron was measured axially at the 249.772 nm wavelength and the internal standard Beryllium was measured axially at the 313.042 nm wavelength. Beryllium was tee-ed into the solution at 1:5 the flowrate before introduction to the spray chamber. A standard curve using 1000, 100, 10, 1 and 0 ppb of boron was used to calculate the concentration of boron in each sample. Once determined, the boron measurement for each sample was normalized to protein content and final boron uptake results were expressed as ng boron/mg protein.

As can be seen in FIG. 15(A), the dipeptide BPA-BPA was taken up by several cancer cell lines; to a similar extent in mouse tumor line CT26 and human tumor line T98G, but slightly lower than the LAT1 substrate BPA in FaDu and A431 cancer cell lines that both highly express LAT1. In FIG. 15(B), three (3) additional dipeptides, BPA-Ala, Ala-BPA, and Leu-BPA were taken up by FaDu tumor cells, but also to a lower extent than BPA.

### Example 13: Evaluation of Differential Uptake of BPA and His-BPA in AsPC-1 and FaDu Cell Lines and Relative Expression Correlation

In this experiment, the differential uptake of BPA and His-BPA was evaluated in cells that express LAT1 and PEPT1. FIG. 16(A) shows the Cancer Cell Line Encyclopedia RNA expression data (RNAseq) for the large neutral amino acid transporter LAT1 (SLC7a5) and the dipeptide transporters PEPT1 (SLC15a1) and PEPT2 (SLC15a2) in FaDu and AsPC-1 cancer cell lines. See, sites.broadinstitute.org/ccle. The results show FaDu has high expression of LAT1 but not PEPT1 whereas AsPC-1 has high expression of PEPT1 and moderate expression of LAT1. Both cell lines have low expression of PEPT2.

In another experiment, Immunohistochemical analysis of LAT1 and PEPT1 protein expression in FaDu and AsPC-1 cancer cells was evaluated using the following protocols. Briefly, FaDu and ASPC-1 cell pellet samples were fixed in 10% neutral buffered formalin, processed, embedded in paraffin wax, and prepared as 4 µm sections mounted on microscope slides. After deparaffinization and re-hydration cell pellet sections were treated for antigen retrieval. The antigen epitopes in FaDu and ASPC-1 were retrieved by the application of heat and pressure by immersion in Diva Decloaker (a pH 6.2 heat-induced epitope retrieval buffer, Biocare LLC) for PEPT1 and Borg Decloaker (a pH 9.5 heat-induced epitope retrieval buffer, Biocare LLC) for LAT1 for 15 minutes at 110° C. in a pressure cooker (Decloaking Chamber, Biocare LLC).

After antigen retrieval antibodies to PEPT1 (rabbit polyclonal antibody from Sino Biological, Catalog No. 203418-T10) and LAT1 (rabbit monoclonal antibody EPR17573 from Abcam, Catalog No. ab208776) were applied to the cell pellet sections, followed by goat anti-rabbit Ig horseradish peroxidase polymer (MACH4 Universal HRP, Biocare LLC) which binds to the PEPT1 or LAT1 rabbit antibody. Subsequent application of the chromogen 3, 3′-diaminobenzidine (DAB) to the cell pellet sections reacts with the horseradish peroxidase to produce a brown colored product at the site of the antigen. The stained cell pellet sections were scanned, and images taken.

The results show that the protein expression levels of LAT1 and PEPT1 correlates with the RNA data presented. FaDu exhibits an elevated level of staining for LAT1 but little staining for PEPT1. In contrast ASPC-1 exhibits an elevated level of PEPT1 staining but little staining for LAT1. (See, FIG. 17(B)).

In another experiment, uptake of BPA and dipeptide His-BPA was carried out in FaDu, and ASPC-1 as described in FIG. 15. FaDu cells expressing elevated levels of LAT1 took up BPA to a much greater extent than His-BPA. In contrast, His-BPA was taken up to a higher level than BPA in PEPT1 high expressing AsPC1 cells. Accordingly, the data supports the finding that borylated dipeptides may be taken up efficiently in PEPT1 or other dipeptide transporter expressing cancers. See, FIG. 16(C).

### Example 14: Evaluation of His-BPA Uptake Inhibition by Gly-SAR in PEPt1 Cells

In this experiment, uptake of BPA and dipeptide His-BPA (0.8 mM of each) was carried out in FaDu and ASPC-1 in the presence and absence of the indicated concentrations of PEPT1 dipeptide transporter competitive inhibitor Gly-SAR. Normalized boron uptake was determined as in FIG. 15.

The results show, Gly-SAR had a dose dependent inhibitory effect on His-BPA uptake in ASPC-1 cells but had no effect in FaDu cells. This supports the proposition that PEPT1 mediates the higher level of His-BPA uptake in ASPC-1 compared to FaDu cells. Additionally, Gly-SAR had no effect on BPA uptake in either cell line supporting the proposition that LAT1 is the predominant transporter for, BPA uptake in both cell lines.

The resulting observation that His-BPA does deliver a boron signal in PEPT1 negative FaDu cells supports the proposition of the existence of another uptake mechanism for His-BPA in this cell line and potentially other PEPT1 low or negative cancers. See, FIG. 18.

### Example 15: Evaluation of Relative Uptake of Dipeptides in PEPT1 Cells

In this experiment, the uptake of several dipeptide and dipeptide analogs was carried out in FaDu and ASPC-1 cells. Normalized boron uptake was determined as in FIG. 15. The results show that all dipeptides and analogs exhibited higher uptake in ASPC-1 than in FaDu cells, which confirms the proposition that PEPT1 is the transporter mediating most uptake. However, several dipeptides are still taken up in PEPT1 negative FaDu cells, although to a lower extent, which further confirms the proposition that an uptake mechanism does exist in FaDu cells for these dipeptides that is not PEPT1. See, FIG. 19.

### Example 16: Evaluation of Dipeptide Degradation to BPA by HPLC

In this experiment, to investigate whether dipeptides are converted to BPA, which is then taken up through its cognate transporter, evaluation of whether dipeptides remained intact in the tissue culture media in the presence of absence of cancer cells was made using the following protocols. Briefly, FaDu cells were harvested and counted. The cell number was adjusted to 50×10⁵ / mL in the HBSS tissue culture medium using 1 mL per well. The cells were incubated at 37° C. in the 5% CO2 overnight. At the end, the supernatants were aspirated, and cells were rinsed with 1 mL PBS once. Each test article was prepared at 2.5 mM concentration in HBSS medium, and 0.4 mL was added to cells. After 4 hrs. at 37° C. the supernatants were harvested and analyzed by RP HPLC using an ACQUITY BEH C18 column 2.1 × 50 mM (Waters) equipped with a matching guard column equilibrated with 2% aqueous acetonitrile/0.1% formic acid mobile phase. The dipeptides were separated in the gradient of 90% acetonitrile/10% water/0.1% formic acid to 20% in 6 min and identified by UV225 nm. The masses were confirmed by an online QDA mass spectrometer. Breakdown of the original dipeptide was quantitated by comparing with the control - the same dipeptide incubated in the same fashion in the absence of cells. Stabilities of BPA-BPA, Ala-BPA, and BPA-Ala were compared, and the formation of BPA was measured semi-quantitatively by RP-HPLC/MS.

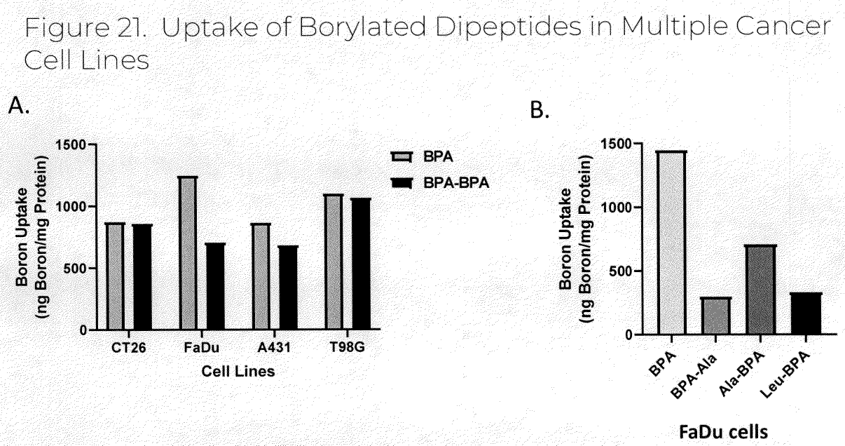

The results in FIG. 20(A) shows a representative chromatogram at 225 nm showing that BPA (peak 1) is formed from BPA-BPA (peak 2) upon incubation with cells for 4 hrs. Only trace amount of BPA is detected in the absence of cells. See, FIG. 20(B). The results in FIG. 20(C) show MS confirmation of the peak assignment. LC/MS confirmation of BPA: [M+H] for BPA is 210 and [M+H] for BPA-BPA is 400. FIG. 21(D) shows the conversion into BPA of 3 dipeptides in the presence and absence of FaDu cells. The relative conversion in the presence of cells is Ala-BPA > BPA-BPA > BPA-Ala.

### Example 17: Pharmacokinetics of BPA-BPA

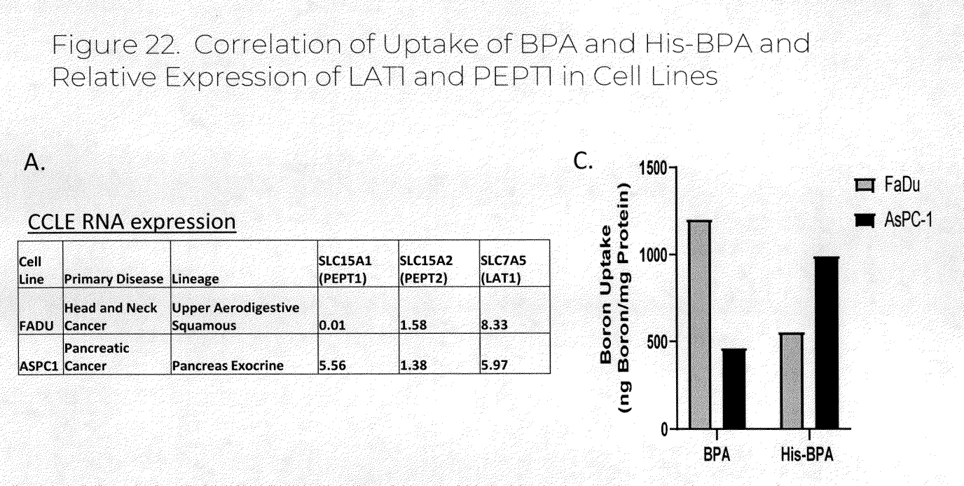

In this experiment, pharmacokinetics of BPA-BPA was evaluated using the following protocols. Briefly, 200 mg/mL of each compound was injected in the tail vein of Balb/C non-tumor bearing male mice (5 mice per group). Blood was drawn at the following time points: 2, 5, 16, 30, 60, 120 and 240 minutes into EDTA-coated tubes. Boron concentration was measured using ICP OES following the digestion in concentrated nitric acid for 1 hour. The boron concentration was normalized to 1 mL of blood and plotted using GraphPad Prizm. The PK parameters (see, FIG. 22, Table) were obtain using PK Solver ver. 2.0 using compartmental analysis BPA-fructose was used as a reference substance.

The results show, BPA-BPA exhibited bi-phasic pharmacokinetics with remarkably similar t_{½} beta (e.g., elimination phase) and clearance (CL) to BPA (the standard of care in BNCT). Generally, it takes longer for BPA-BPA to clear from the system (218 min versus 195 min for BPA). The volume of distribution at the steady state (Vss) is similar to that of BPA which confirms the proposition that the dipeptide has a similar blood protein binding and is readily accessible to eliminating organs. The maximum plasma concentrations are similar for both test articles. However, the total drug exposure across time (AUC from zero to infinity) is marginally lower for BPA-BPA. See, FIG. 22.

### Example 18: Biodistribution of Dipeptides Using FaDu Cells In Vivo

In this experiment, biodistribution of various dipeptides were evaluated in vivo using the following protocols. All animal studies were carried out following the “Guide for the Care and Use the Laboratory Animals,” 8^{th} Ed. and Animal Welfare Act (USDA). Human hypopharyngeal squamous cell carcinoma FaDu cells were maintained in DMEM, supplemented with L-glutamine and 10% FBS.

Subcutaneous (s.c. or sc) tumors were generated by injection of 2.5 × 10⁶ cancer cells mixed at a 1:1 dilution with Matrigel (Corning Life Sciences) in the right flank of female CB17 SCID mice. Tumor sizes were determined by caliper measurements, and the tumor volume was calculated as width²×Length/2, wherein width is the smallest dimension and length is the largest dimension. Tumors were allowed to grow untreated until they reached an approximate volume of 300 mm³. At that point, animals were randomized and allocated to each treatment group based on tumor volume at the time of treatment initiation to ensure similar mean tumor size and variation in each group. Each group received a single dose of BPA at 200 mg/mg or a test article at 800 mg/mg via intravenous tail vein injection. The injection volume did not exceed 200 µL per mouse in concord with veterinary guidelines. Two hours post dose, blood was collected from each mouse from the submandibular vein into K2 EDTA coated tubes. Mice were then humanely euthanized, and tumor and organs were collected for boron analysis.

Furthermore, all compounds tested, with the exception of BPA were prepared as 80-100 mg/mL stocks. BPA was prepared as a fructose solution at 20 - 22 mg/mL. The concentrations were confirmed by ICP OES prior to the study. The blood and tissues (tumor, kidney, and pancreas) were harvested at the times indicated, weighed, and placed inside the Teflon containers and digested using the CEM microwave oven. The digested tissues were analyzed by ICP OES to determine the boron concentration.

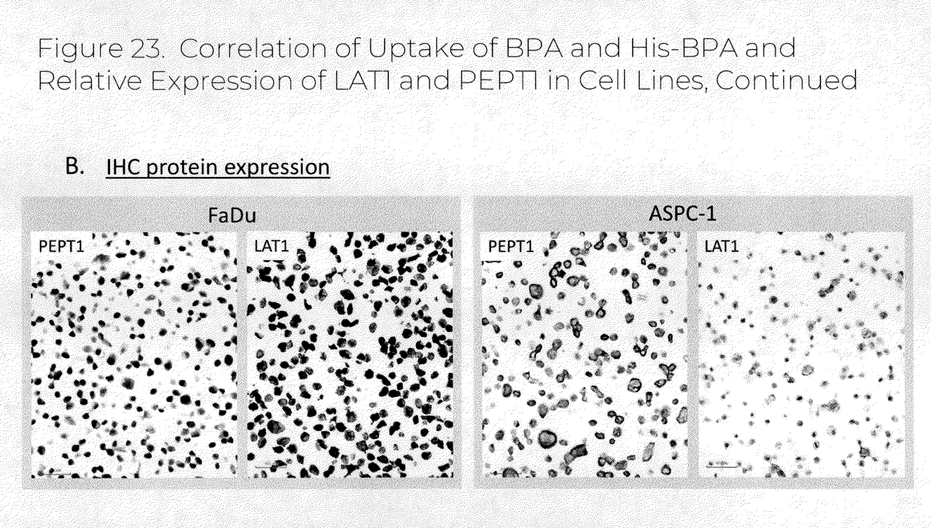

The results show that the four (4) dipeptides tested were dosed at a concentration 4X higher than that used for BPA-F due to the much higher solubility of these dipeptides. The amounts of boron delivered to the tumors at one (1) hr. time point by the dipeptides were 2.1-2.7 × higher to the boron amounts observed in the tumors of the mice dosed with BPA-F at 200 mg/kg. It is noted that the highest boron uptake was observed for Ala-BPA-F (53.9 µg boron/g tumor as shown on the Inset). Significantly higher amounts of boron are observed in the kidneys and pancreas of all of the mice dosed with the dipeptides when compared to BPA-F. See, FIG. 23.

### Example 19: Biodistribution of His-BPA Using FaDu Cells In Vivo

In this experiment, biodistribution of His-BPA was evaluated in vivo using the following protocols. All animal studies were carried out following the “Guide for the Care and Use the Laboratory Animals,” 8^{th} Ed. and Animal Welfare Act (USDA). Human hypopharyngeal squamous cell carcinoma FaDu cells were maintained in DMEM, supplemented with L-glutamine and 10% FBS.

Subcutaneous (s.c.) tumors were generated by injection of 2.5 × 10⁶ cancer cells mixed at a 1:1 dilution with Matrigel (Corning Life Sciences) in the right flank of female CB17 SCID mice. Tumor sizes were determined by caliper measurements, and the tumor volume was calculated as width²×Length/2, wherein width is the smallest dimension and length is the largest dimension. Tumors were allowed to grow untreated until they reached an approximate volume of 300 mm³. At that point, animals were randomized and allocated to each treatment group based on tumor volume at the time of treatment initiation to ensure similar mean tumor size and variation in each group. Each group received a single dose of BPA at 200 mg/mg or a test article at 800 mg/mg via intravenous tail vein injection. The injection volume did not exceed 200 µL per mouse in concord with veterinary guidelines. Two hours post dose, blood was collected from each mouse from the submandibular vein into K2 EDTA coated tubes. Mice were then humanely euthanized, and tumor and organs were collected for boron analysis.

Furthermore, all compounds tested, with the exception of BPA were prepared as 80-100 mg/mL stocks. BPA was prepared as a fructose solution at 20 - 22 mg/mL. The concentrations were confirmed by ICP OES prior to the study. The blood and tissues (tumor, kidney, and pancreas) were harvested at the times indicated, weighed, and placed inside the Teflon containers and digested using the CEM microwave oven. The digested tissues were analyzed by ICP OES to determine the boron concentration.

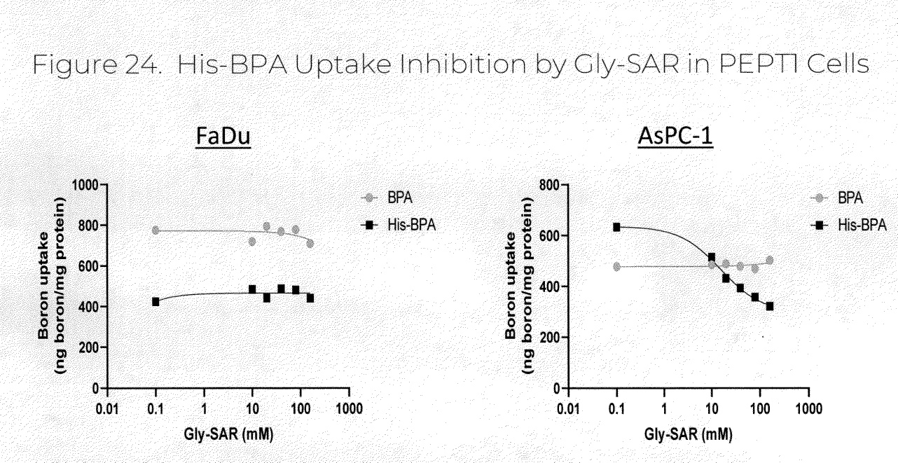

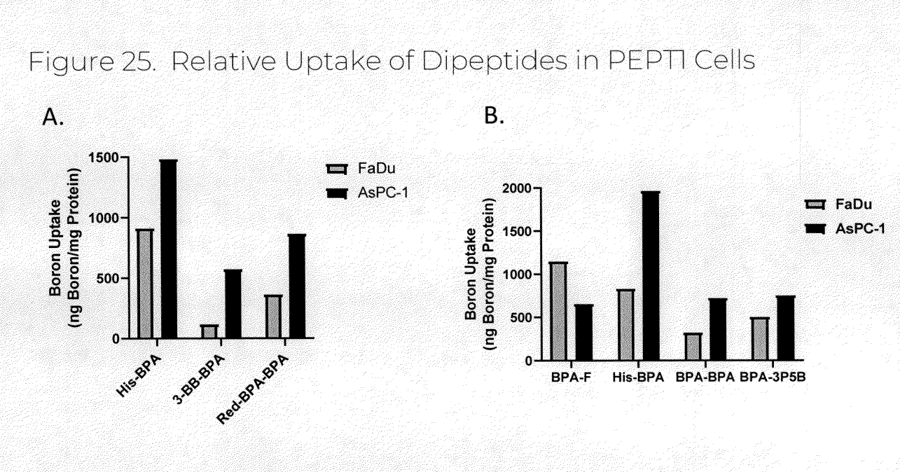

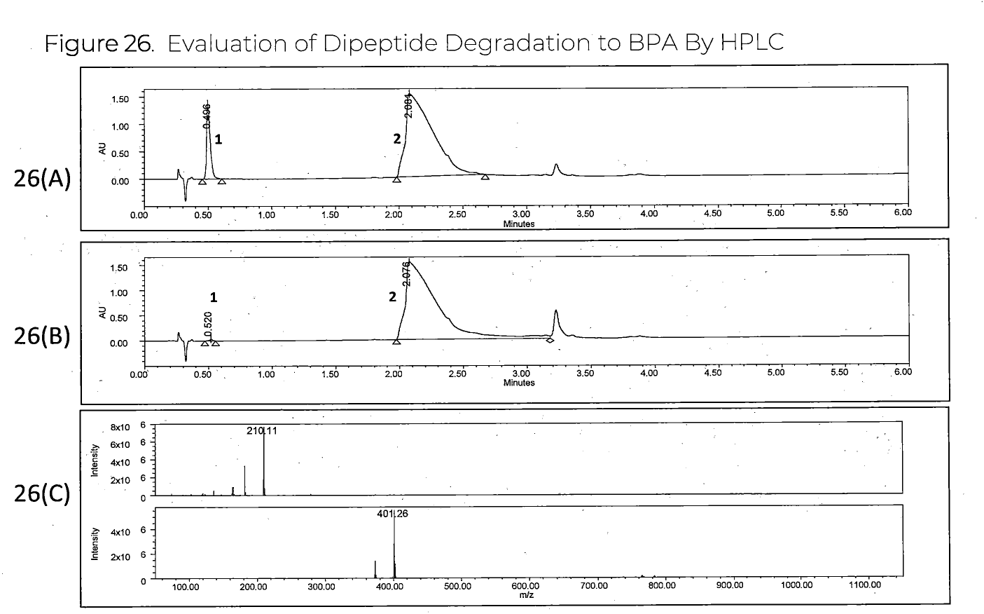

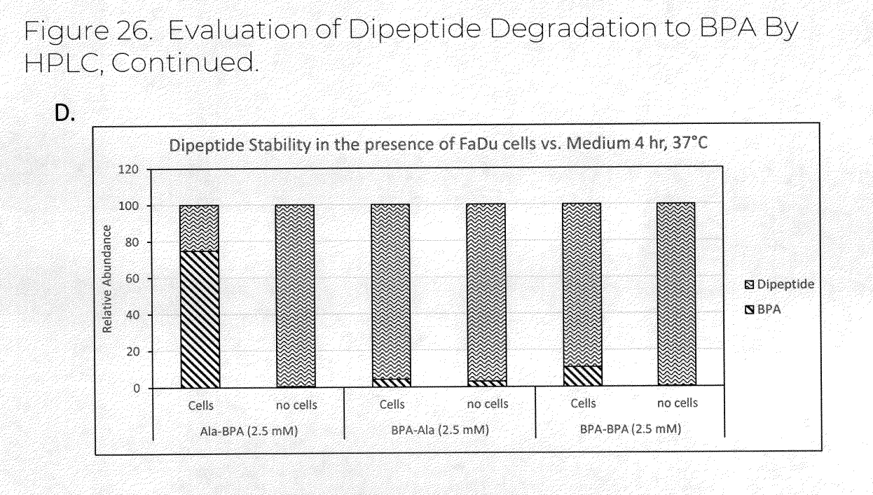

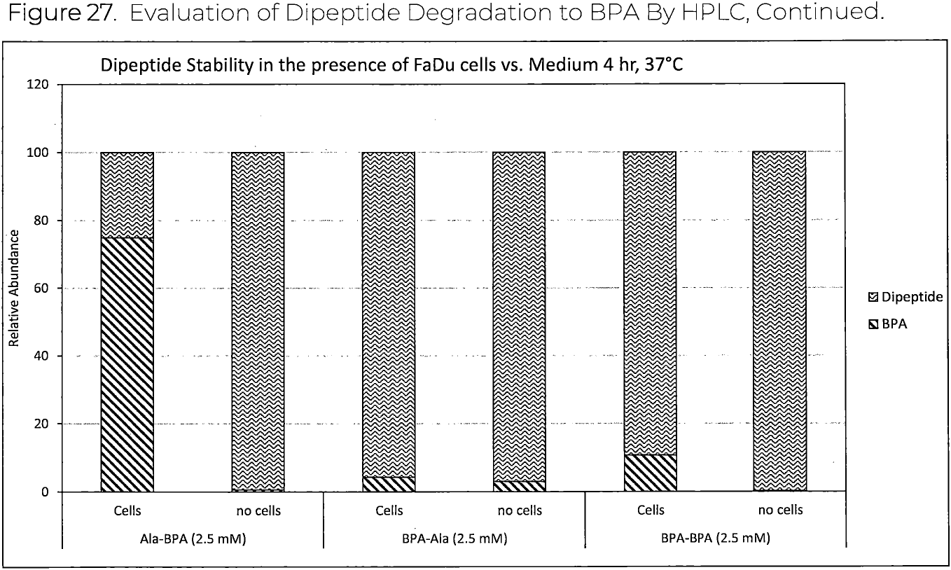

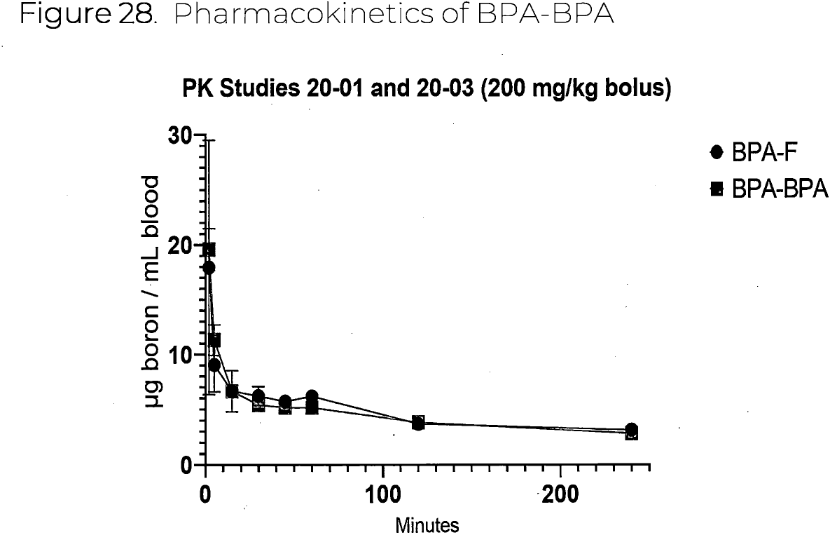

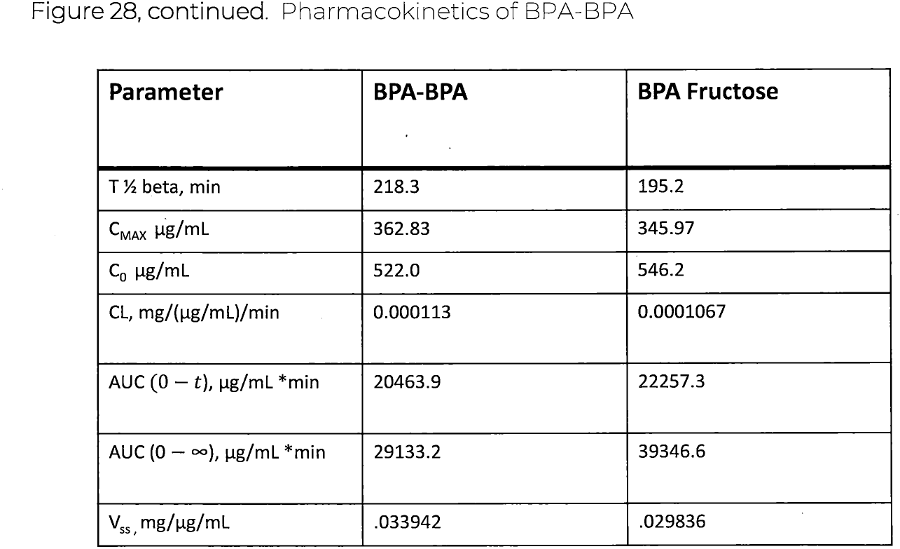

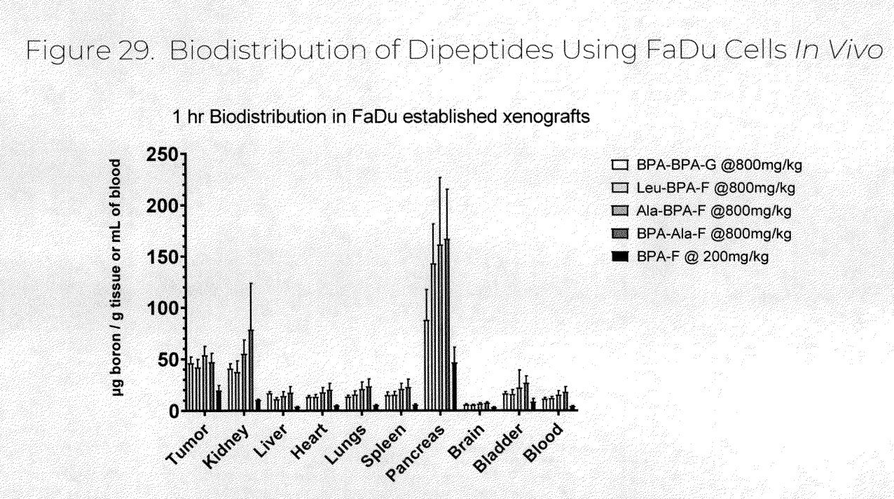

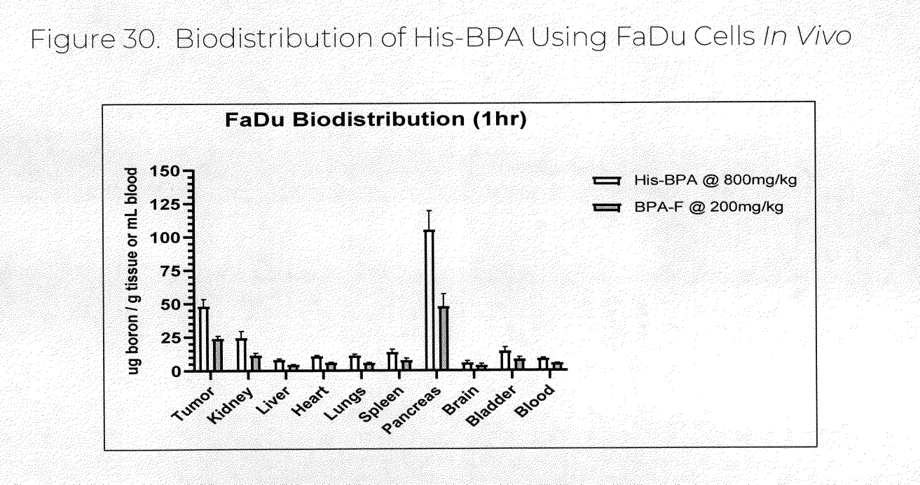

The results show, His-BPA was able to be dosed at a concentration 4X higher than that used for BPA-F, similar to the other dipeptides. This was due to the high solubility of this dipeptide. The amounts of boron delivered to the tumors by this dipeptide was 2x higher than the boron level in the tumors from mice dosed with BPA-F at 200 mg/kg. This uptake is similar to that observed for the other dipeptides tested. Significantly higher amounts of boron are observed in the kidneys and pancreas in all of the mice dosed with His-BPA when compared to BPA-F. See, FIG. 24.

### Example 20: Dose Escalation of Boron Delivery Using Borylated Dipeptides in Multiple Xenograft Models In Vivo

In this experiment, FaDu and Detroit-562 (head and neck), MeWo (melanoma), HCC-1954 (breast), A549 (lung), and AsPC-1 (pancreatic) human cancer xenograft models implanted in CB17 SCID mice were treated with 200 or 400 mg/kg of BPA, or because of improved solubility, His-BPA, or BPA-BPA at 1000 mg/kg. Tumors were harvested 120 minutes following treatment and boron content determined by ICP-OES. Data are the means ± SD of 5 mice per treatment group.

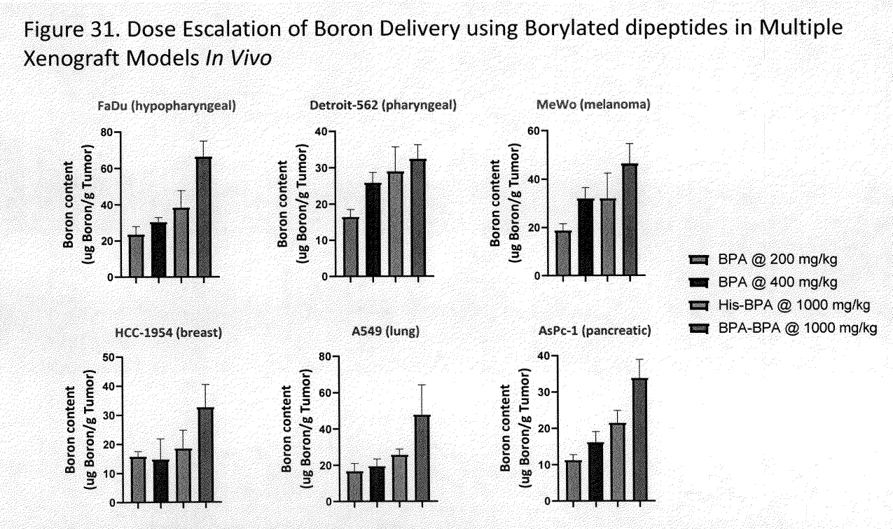

The results show that both dipeptides, were able to be dosed to 1000 mg/kg, compared to a maximal dose of 400 mg/kg for BPA. BPA-BPA dosed at 1000 mg/kg, with 2 boron atoms per molecule, was able to deliver the greatest boron to all tested xenografts, achieving up to 60 ug/g of tumor in FaDu xenografts and a minimum of just over 30 ug/g in Detroit-562, HCC-1954, and ASPC-1 models, and just over 40 ug/g in MeWO and A549 models. In addition, His-BPA, dosed at 1000 mg/kg delivered higher boron levels than BPA dosed at standard 200 mg/kg, and similar levels compared to a maximal, but non-storable without precipitation, BPA dose of 400 mg/kg. See, FIG. 31.

### Example 21: Assessing Tumor Boron Content of Multiple Dipeptides Using CT26 Syngeneic Colon Cancer Model

In this experiment, CT26 murine syngeneic colon cancer xenograft models implanted in BALB/c mice were treated with 200 mg/kg of BPA as well as His-BPA and BPA-BPA at both 200 and 800 mg/kg, respectively. Tumors were harvested 120 minutes following treatment and boron content determined by ICP-OES. Data are the means ± SD of 5 mice per treatment group.

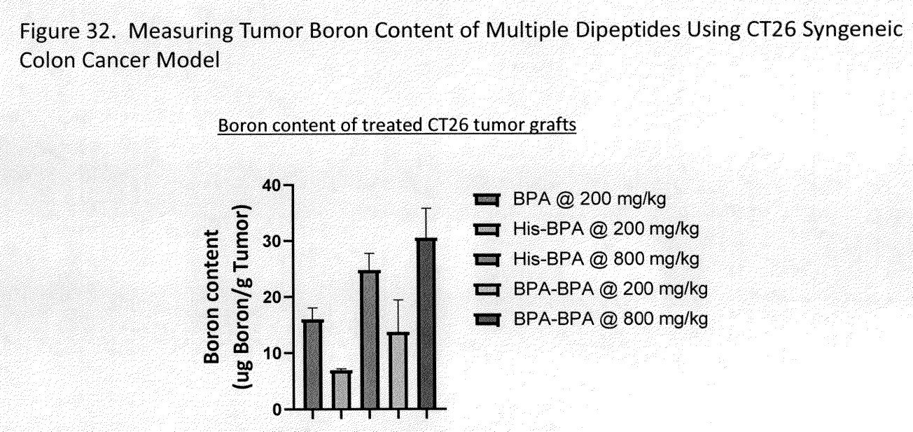

The results show that His-BPA and BPA-BPA, both dosed at 800 mg/kg, delivered 25 ug/g boron and 30 ug/g of tumor respectively, compared to ~16 ug/g tumor for BPA dosed at 200 mg/kg. These results suggest that there is greater efficacy of dipeptide BNCT over BPA, and optimally more efficacy of dipeptides with a smaller neutron dose. See, FIG. 32.

### Example 22: BNCT Studies Using Borylated Dipeptides Compared to BPA

In this experiment, CT26 xenografts in BALB/c mice were dosed with 200 mg/kg of BPA or, because of improved solubility, 800 mg/kg of His-BPA or BPA-BPA. Following one (1) hour of treatment time, mice were irradiated for either 12 minutes (33(A)) or 6 minutes (33(B)) at the Kyoto University Research Reactor (Kyoto, Japan) 1 set at 5 megawatts. Data are the means ± SD of 3 mice per treatment group. Tumors were measured for growth out to 22 days.

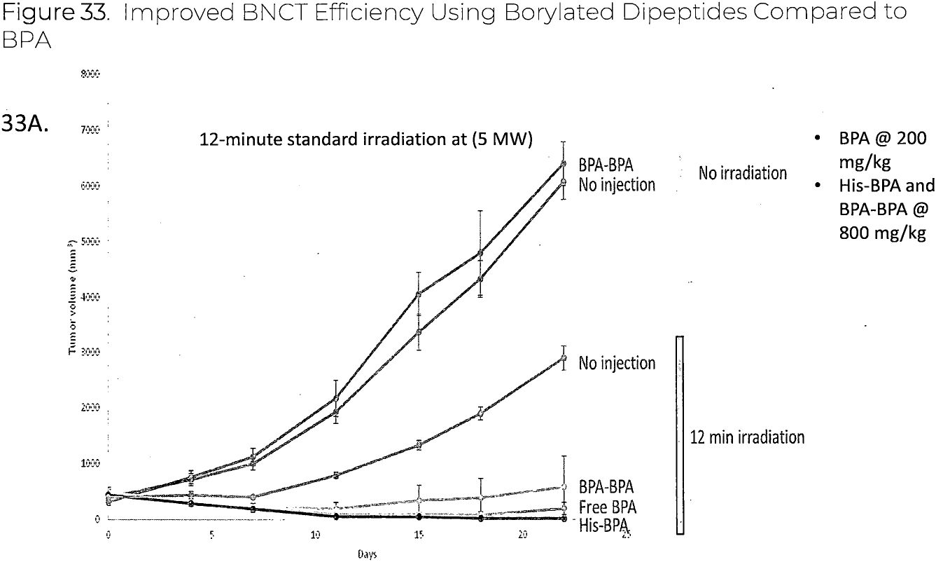

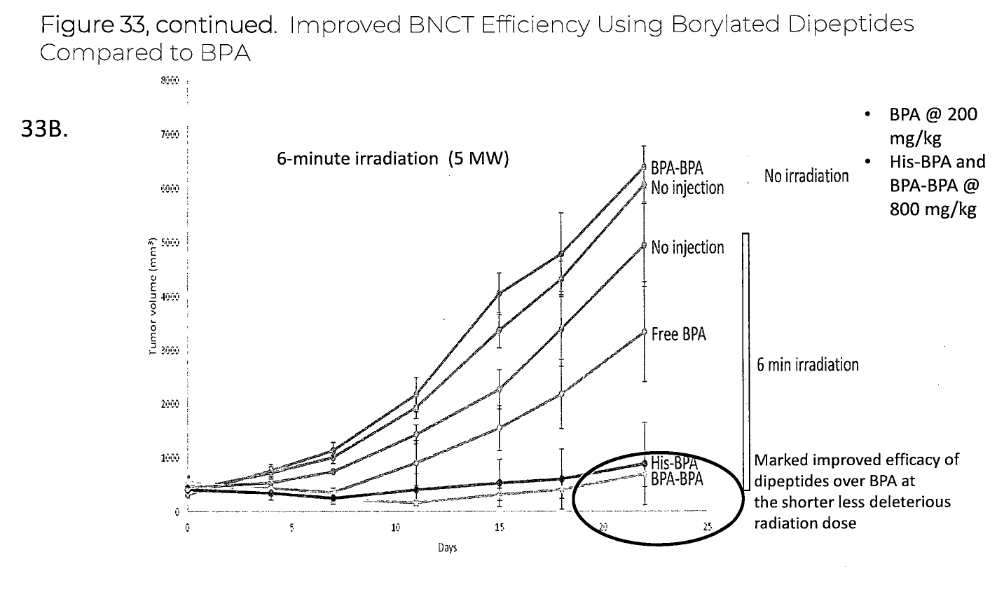

As can be seen in FIG. 33(A), irradiation alone had a moderate anti-tumor growth effect that is indicative that normal tissue may also be affected at this high irradiation dose. All 3 compounds, BPA, His-BPA, and BPA-BPA, however, did have a significant and similar growth inhibition, even regression, when monitored out to 22 days. Accordingly, since dipeptides delivered higher amounts of boron compared to BPA in the tumor biodistribution experiment (FIG. 32), then based on a shorter irradiation time of 6 minutes (lower total neutron dose), the dipeptides should demonstrate a significantly greater anti-tumor effect than BPA.

The results in FIG. 33(B) confirmed this fact. As can be see, irradiation alone was similar to the no irradiation control, BPA with irradiation mediated only modest tumor inhibition, however, both dipeptides mediated almost complete tumor inhibition monitored out to 22 days.

### Example 23: BNCT Studies Showing Tumor Regression Using Borylated Dipeptides

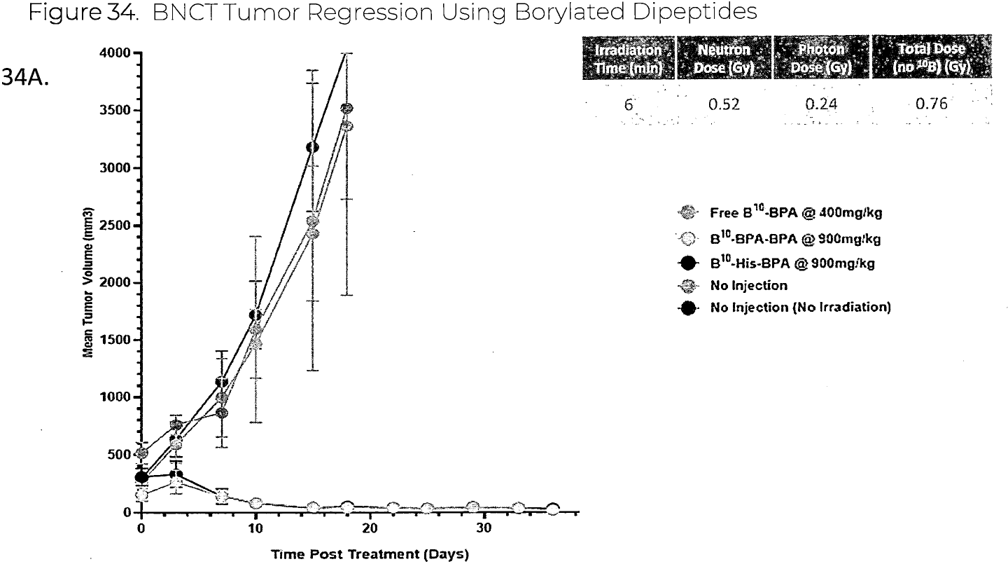

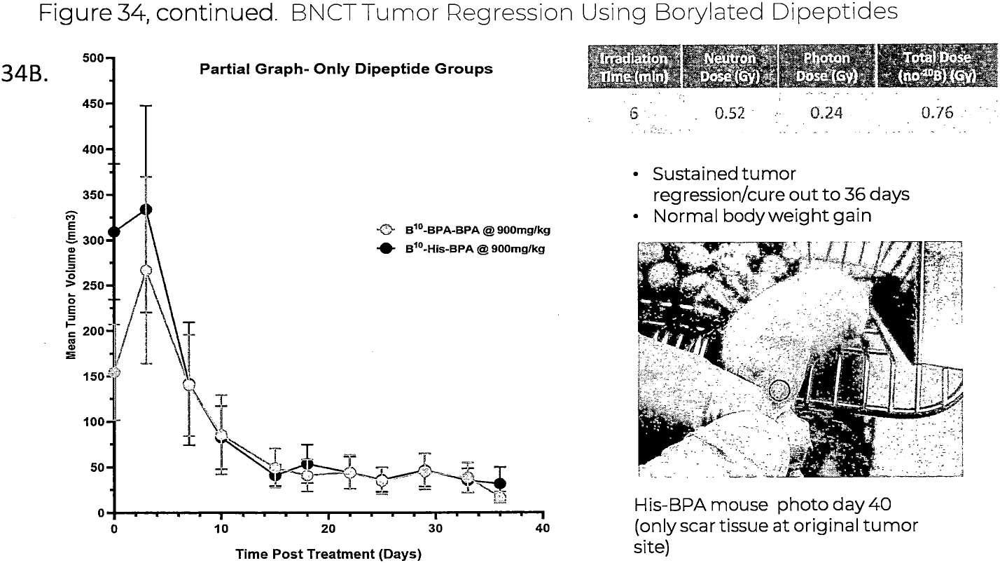

In this experiment, CT26 xenografts in BALB/c mice were dosed with 400 mg/kg of BPA or 900 mg/kg of His-BPA or BPA-BPA. Following one (1) hour treatment time, mice were irradiated for 6 minutes at the Kyoto University Research Reactor (Kyoto, Japan) 1 set at 5 megawatts. Data are the means ± SD of 3 mice per treatment group. Tumors were measured for growth out to 36 days and examined for tumor presence at 40 days. The graph presented in FIG. 34(B) is a magnified version of the growth curves for the dipeptide groups presented in FIG. 34(A). BPA and control groups were sacrificed at day 18 due to excessive tumor growth.

FIG. 34 shows a BNCT experiment comparing the efficacy of BPA against His-BPA and BPA-BPA using a shorter six (6) minute irradiation time (See, Example 22). In this experiment, all control groups, and BPA with irradiation all had continued tumor growth requiring sacrificing of the groups by day 18.

In contrast, His-BPA, and BPA-BPA, dosed at 900 mg/kg with irradiation, had significant tumor regression that was sustained out to 36 days of growth monitoring. Visual inspection and histological examination of the tumor area of a His-BPA treated mouse showed lack of tumor tissue and only scar tissue at the original tumor site, suggestive of actual tumor cures following BNCT treatment. FIG. 33(B).

### Example 24: Human Clinical Trials for the Treatment of Human Carcinomas Through the Use of Bdi-AAs

Bdi-AAs are synthesized in accordance with the present invention which specifically accumulate in a tumor cell and are used in the treatment of certain tumors and other immunological disorders and/or other diseases. In connection with each of these indications, two clinical approaches are successfully pursued.

I.) Adjunctive therapy: In adjunctive therapy, patients are treated with Bdi-AAs in combination with a chemotherapeutic or pharmaceutical or biopharmaceutical agent or a combination thereof. Primary cancer targets are treated under standard protocols by the addition of Bdi-AAs and then irradiated. Protocol designs address effectiveness as assessed by the following examples, including but not limited to, reduction in tumor mass of primary or metastatic lesions, increased progression free survival, overall survival, improvement of patient’s health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.

II.) Monotherapy: In connection with the use of the Bdi-AAs in monotherapy of tumors, the Bdi-AAs are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patient’s health, disease stabilization, as well as the ability to reduce usual doses of standard chemotherapy and other biologic agents.

### Dosage

Dosage regimens may be adjusted to provide the optimum desired response. For example, a single Bdi-AA injection may be administered, several divided doses may be administered overtime, or the dose may be proportionally reduced or increased as indicated by the exigencies of the therapeutic situation. “Dosage Unit Form” as used herein refers to physically discrete units suited as unitary. dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the Bdi-AA, the individual mechanics of the irradiation mechanism (reactor) and the particular therapeutic or prophylactic effect to be achieved, and (b) the limitations inherent in the art of compounding such a compound for the treatment of sensitivity in individuals.

### Clinical Development Plan (CDP)

The CDP follows and develops treatments of cancer(s) and/or immunological disorders using Bdi-AAs of the disclosure which are then irradiated using Neutron Capture Therapy in connection with adjunctive therapy or monotherapy. Trials initially demonstrate safety and thereafter -confirm efficacy in repeat doses. Trials are open label comparing standard chemotherapy with standard therapy plus Bdi-AAs which are then irradiated using Boron Neutron Capture Therapy. As will be appreciated, one nonlimiting criteria that can be utilized in connection with enrollment of patients is concentration of Bdi-AAs in a tumor as determined by standard detection methods known in the art.

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I

Naturally Occuring Amino Acids

| | | |
|---|---|---|
| SINGLE LETTER | THREE LETTER | FULL NAME |
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

1. A composition comprising a chemical structure as follows: 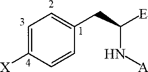
whereby A = H, Amino Acid, or Borylated Amino Acid;
E = CO₂H, B(OH)₂, CO-Amino Acid, or CO-Borylated Amino Acid, CO-NHB₁₂H_{11;} and
X = H, B(OH)₂, BF₃K, or B(OR)₂, or can be at position 2, 3, or 4.

2. A composition comprising a chemical structure as follows: 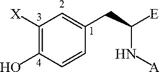
whereby A = H, Amino Acid, or Borylated Amino Acid;
E = CO₂H, B(OH)₂, CO-Amino Acid, or CO-Borylated Amino Acid, CO-NHB₁₂H₁₁; and
X = H, B(OH)₂, BF₃K, or B(OR)₂, or can be at position 2, or 3.

3. A composition comprising a chemical structure as follows: 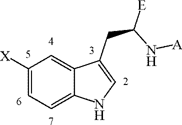
whereby A = H, Amino Acid, or Borylated Amino Acid;
E = CO₂H, B(OH)₂, CO-Amino Acid, or CO-Borylated Amino Acid, CO-NHB₁₂H₁₁; and
X = H, B(OH)₂, BF₃K, or B(OR)₂, or can be at position 2, 4, 5, 6, or 7.

4. A kit comprising the composition of claim 1.

5. A kit comprising the composition of claim 2.

6. A kit comprising the composition of claim 3.

7. A method of producing a composition of claim 1.

8. A method of producing a composition of claim 2.

9. A method of producing a composition of claim 3.

10. A Dosage Unit form comprising a composition of claim 1.

11. A Dosage Unit form comprising a composition of claim 2.

12. A Dosage Unit form comprising a composition of claim 3.

13. A method of performing Neutron Capture Therapy in the treatment of human cancer comprising:
synthesizing a Human Unit Dose of a borylated di-peptide amino acid (Bdi-AA) composition;
njecting the Bdi-AA into a tumor, whereby the Bdi-AA accumulates into a cell; and
irradiating the Bdi-AA with neutrons.

14. The method of claim 13, wherein the composition is selected from the group consisting of the compositions in claim(s) 1, 2, and 3.

15. The method of claim 13, wherein the Neutron Capture Therapy is Boron Neutron Capture Therapy.

16. The method of claim 13, wherein the Neutron Capture Therapy is Proton Boron Fusion Therapy.

17. The method of claim 15, wherein the irradiation comprises epithermal neutrons.

18. The method of claim 13, wherein the cancer is colon cancer.

19. The method of claim 13, wherein the cancer is head and neck cancer.

20. The method of claim 13, wherein the cancer is pancreatic cancer.
